(19) United States
(12) Patent Application Publication
(10) Pub. No.: US 2015/0299698 A1
(43) Pub. Date: Oct. 22, 2015

(54) METHODS FOR ENGINEERING NON-NEURONAL CELLS INTO NEURONS AND USING NEWLY ENGINEERED NEURONS TO TREAT NEURODEGENERATIVE DISEASES

(71) Applicant: The Regents of the University of California, Oakland, CA (US)

(72) Inventors: Xiang-Dong Fu, La Jolla, CA (US); Yuanchao Xue, San Diego, CA (US)

(21) Appl. No.: 14/439,125

(22) PCT Filed: Nov. 1, 2013
(86) PCT No.: PCT/US2013/068005

§ 371 Date: Apr. 28, 2015

Related U.S. Application Data
(60) Provisional application No. 61/721,439, filed on Nov. 1, 2012.

(51) Int. Cl. C12N 15/113; A61K 31/713; A61K 45/06; C12N 5/0793

(52) U.S. Cl. CPC C12N 15/113; C12N 5/0619; A61K 31/713; A61K 45/06; C12N 2501/998; C12N 2310/11; C12N 2320/30; C12N 2501/20; C12N 2310/531; C12N 2501/15; C12N 2501/2318; C12N 2501/24

(57) ABSTRACT

The invention provides compositions and in vivo, ex vivo and in vitro methods for trans-differentiation of or re-programming mammalian cells to functional neurons. In particular, the invention provides methods for engineering non-neuronal cells into neurons, including fully functional human neuronal cells, and methods for engineering non-neuronal cells into neurons, e.g., fully functional human neuronal cells, in the brain to treat a neurodegenerative disease. In alternative embodiments, the invention provides compositions comprising re-differentiated or re-programmed mammalian cells, such as human cells, of the invention. The invention also provides compositions and methods for direct reprogramming of cells to a second phenotype or differentiated phenotype, such as a neuron, including a fully functional human neuronal cell. The invention also provides formulations, products of manufacture, implants, artificial organs or tissues, or kits, comprising a trans-differentiated or re-programmed cell of the invention, e.g., a fully functional human neuronal cell.

### RELATED APPLICATIONS

This Patent Convention Treaty (PCT) International Application claims the benefit of priority under 35 U.S.C. §119(e) of U.S. Provisional Application No. 61/721,439, filed Nov. 1, 2012. The aforementioned application is expressly incorporated herein by reference in its entirety and for all purposes.

### GOVERNMENT RIGHTS

This invention was made with government support under grants GM049369 and HG004659, awarded by the National Institutes of Health (NIH). The government has certain rights in the invention.

### TECHNICAL FIELD

This invention relates to cellular and developmental biology and regenerative medicine. The invention provides compositions and in vivo, ex vivo and in vitro methods for trans-differentiation of, re-differentiating or re-programming mammalian cells to functional neurons. In particular, the invention provides methods for engineering non-neuronal cells into neurons, and methods for engineering non-neuronal cells into neurons in the brain to treat a neurodegenerative disease. In alternative embodiments, the invention provides compositions comprising re-differentiated or re-programmed mammalian cells of the invention. The invention also provides compositions and methods for direct reprogramming of cells to a second phenotype or differentiated phenotype, such as a neuron. The invention also provides formulations, products of manufacture, implants, artificial organs or tissues, or kits, comprising a trans-differentiated or re-programmed cell of the invention.

### BACKGROUND

Neuronal differentiation is a well-studied paradigm as a consequence of transcription reprogramming Recent studies have shown that a set of neuronal lineage-specific transcription factors is sufficient to trans-differentiate fibroblasts into functional neurons. Neuronal differentiation is subject to additional layers of control, such as regulated RNA processing.

The Polypyrimidine Tract Binding protein, PTB and its homolog “neuronal PTB” or nPTB, undergo a programmed switch during neuronal differentiation. Homeostatic expression of PTB in non-neuronal cells is maintained through splicing auto-regulation. When PTB is down regulated by miR-124, an internal alternative exon is included, rendering the transcript sensitive to nonsense mediated RNA decay, thereby re-enforcing PTB down-regulation. Reduced PTB also results in increased nPTB expression and forced expression of PTB blocks miR-124 induced neuronal differentiation. However, it has been unclear whether the PTB/nPTB switch is sufficient to initiate neuronal differentiation and which specific PTB/nPTB-regulated splicing events contribute to the cell fate switch.

### SUMMARY

In alternative embodiments, the invention provides in vitro, ex vivo or in vivo methods for trans-differentiating, re-differentiating or re-programming a mammalian cell to a neuronal cell, comprising:
(a) (i) providing a composition or compound for:
reducing or lowering the level of expression of or activity of or inactivating a Polypyrimidine Tract Binding protein (PTB) gene, message or protein;
reducing or lowering the level of expression of or activity of or inactivating a “neuronal PTB homologue”, or nPTB, gene, message or protein; or,
reducing or lowering the level of expression of or activity of or inactivating an RE1-Silencing Transcription factor (REST; also known as Neuron-Restrictive Silencer Factor, or NRSF) complex;
(ii) providing a non-neuronal mammalian cell; and
(iii) contacting in vitro, ex vivo or in vivo the composition or compound with the non-neuronal mammalian cell in an amount effective to cause the trans-differentiating, re-differentiating or re-programming of the mammalian cell to a neuronal cell;
(b) the method of (a), wherein the composition or compound comprise a protein, a peptide, an antibody, a nucleic acid, an antisense or miRNA nucleic acid, or a small molecule;
(c) the method of (b), wherein the antisense or miRNA nucleic acid comprises a neuronal-specific miR-124;
(d) the method of (a) or (b), wherein the method comprises the sequential reducing or lowering the level of expression of or activity of or inactivating of first PTB, and then nPTB, in the mammalian cell to be trans-differentiated, re-differentiated or re-programmed to a neuronal cell,
wherein optionally the mammalian cell to be trans-differentiated, re-differentiated or re-programmed to a neuronal cell is a fibroblast;
and optionally the mammalian cell and the neuronal cell are human cells;
and optionally the sequential reducing or lowering of the level of expression of or activity of or inactivating of first PTB, and then nPTB, in the mammalian cell comprises: waiting at least about 4 days (or between about 1 to 4 days, or between about 1 to 5 days) after the reducing or lowering of the level of expression of or activity of or inactivating of the PTB before the reducing or lowering of the level of expression of or activity of or inactivating of the nPTB,
and optionally the sequential reducing or lowering the level of expression of or activity of or inactivating of first PTB, and then nPTB, in the mammalian cell comprising knocking out the gene for PTB and/or nPTB.
In alternative embodiments, the mammalian cell is: a human cell, a non-human primate cell, a monkey cell, a mouse cell, a rat cell, a guinea pig cell, a rabbit cell, a hamster cell, a goat cell, a bovine cell, an equine cell, an ovine cell, a canine cell or a feline cell; or a fibroblast, or a glial cell.
In alternative embodiments, the composition or compound comprises a or is formulation in or as a liquid or aqueous formulation, a vesicle, liposome, nanoparticle or nanolipid particle, and optionally the in vitro or ex vivo contacting is on mammalian cells embedded in a gel, or the in vitro or ex vivo contacting is on a mammalian cell that is adherent on (to) a plate or a fixed or gel structure.
In alternative embodiments, the mammalian cell is contacted with the composition, or the liquid or aqueous formulation, or the vesicle, liposome, nanoparticle or nanolipid particle, in an amount effective to cause the trans-differentiation or re-programming of the mammalian cell to a neuronal cell.
In alternative embodiments, the mammalian cell before trans-differentiation or re-programming, is an adult stem cell, an embryonic stem cell, a somatic stem cell, an adipose-derived stem cell (ASC), a stem cell derived from an epithelial cell or tissue, a hematopoietic stem cell, a mammary stem cell, a mesenchymal stem cell, a neural stem cell, an olfactory adult stem cell, a spermatogonial progenitor cell, a dental pulp-derived stem cell, or a cancer stem cell, or an adult somatic cell or an adult germ cell, or is a hematopoietic cell, a lymphocyte, a macrophage, a T cell, a B cell, a nerve cell, a neural cell, a glial cell, an astrocyte, a muscle cell, a cardiac cell, a liver cell, a hepatocyte, a pancreatic cell, a fibroblast cell, a connective tissue cell, a skin cell, a melanocyte, an adipose cell, an exocrine cell, a dermal cell, a keratinocyte, a retinal cell, a Muller cell, a mucosal cell, an esophageal cell, an epidermal cell, a bone cell, a chondrocyte, an osteoblast, an osteocyte, a prostate cell, an embryoid body cell, an ovary cell, a testis cell, an adipose tissue (fat) cell, or a cancer cell.
In alternative embodiments, the invention provides the mammalian cell is cultured for between about one to 24 hours, or between about one to two days. In alternative embodiments, the mammalian cell is cultured for between about one to 10 days after the contacting; or, the mammalian cell is cultured before, during and/or after the contacting.
In alternative embodiments, the mammalian cell is also contacted with a cytokine that has a trans-differentiation or re-programming effect on the mammalian cell, wherein optionally the cytokine comprises a transforming growth factor-beta (TGF-beta), interleukin-18 (IL-18, or interferon-γ-inducing factor), adipose complement-related protein or interferon-γ.

In alternative embodiments, the nucleic acid that is inhibitory comprises an miRNA, an siRNA, a ribozyme and/or an antisense nucleic acid.

In alternative embodiments, the identifying and/or isolating the trans-differentiated or re-programmed cell is by a negative selection of cells still expressing a non-neuronal cell marker, or the trans-differentiated or re-programmed cell is identified and/or isolated by fluorescent activated cell sorting (FACS) or affinity column chromatography, or by identification and/or isolation of plasma membrane proteins by mass spectography or chromatography, or by determining the presence or absence of a message (mRNA, transcript) determinative of an undifferentiated or neuronal cell phenotype.

In alternative embodiments, the methods of the invention further comprise implanting the trans-differentiated or re-programmed mammalian cell in or into a vessel, tissue or organ, wherein optionally the trans-differentiated or re-programmed mammalian cell is implanted in or into a vessel, tissue or organ ex vivo or in vivo. In alternative embodiments, the methods of the invention further comprise implanting the trans-differentiated or re-programmed mammalian cell in or into an individual in need thereof, wherein optionally the individual in need thereof has a neurodegenerative disease or an injury to the CNS, brain or spinal cord.

In alternative embodiments, the invention provides trans-differentiated or re-programmed cells made by practicing any method of the invention, wherein the trans-differentiated or re-differentiated or re-programmed cell is: a neuronal mammalian cell, or a fibroblast, or optionally a functional human cell or functional human neuronal cell, and optionally a cell having both the PTB and nPTB gene knocked out. In alternative embodiments, the mammalian cell is a human cell, a non-human primate cell, a monkey cell, a mouse cell, a rat cell, a guinea pig cell, a rabbit cell, a hamster cell, a goat cell, a bovine cell, an equine cell, an ovine cell, a canine cell or a feline cell.

In alternative embodiments, the invention provides methods for treating or ameliorating a neurodegenerative disease or an injury or neurodegenerative condition, comprising:
(a) (i) providing a composition or compound for:
reducing or lowering the level of expression of or activity of or inactivating a Polypyrimidine Tract Binding protein (PTB) gene, message or protein;
reducing or lowering the level of expression of or activity of or inactivating a “neuronal PTB homologue”, or nPTB, gene, message or protein; or,
reducing or lowering the level of expression of or activity of or inactivating an RE1-Silencing Transcription factor (REST; also known as Neuron-Restrictive Silencer Factor, or NRSF) complex;
(ii) providing a non-neuronal mammalian cell; and
(iii) contacting in vitro, ex vivo or in vivo the composition or compound with the non-neuronal mammalian cell in an amount effective to cause the trans-differentiating, re-differentiating or re-programming of the mammalian cell to a neuronal cell;
(b) the method of (a), wherein the composition or compound comprise a protein, a peptide, an antibody, a nucleic acid, an antisense or miRNA nucleic acid, or a small molecule;
(c) the method of (b), wherein the antisense or miRNA nucleic acid comprises a neuronal-specific miR-124;
(d) the method of (a) or (b), wherein the method comprises the sequential reducing or lowering the level of expression of or activity of or inactivating of first PTB, and then nPTB, in the mammalian cell to be trans-differentiated, re-differentiated or re-programmed to a neuronal cell,
wherein optionally the mammalian cell to be trans-differentiated, re-differentiated or re-programmed to a neuronal cell is a fibroblast;
and optionally the mammalian cell and the neuronal cell are human cells;
and optionally the sequential reducing or lowering of the level of expression of or activity of or inactivating of first PTB, and then nPTB, in the mammalian cell comprises: waiting at least about 4 days (or between about 1 to 4 days, or between about 1 to 5 days) after the reducing or lowering of the level of expression of or activity of or inactivating of the PTB before the reducing or lowering of the level of expression of or activity of or inactivating of the nPTB,
and optionally the sequential reducing or lowering the level of expression of or activity of or inactivating of first PTB, and then nPTB, in the mammalian cell comprising knocking out the gene for PTB and/or nPTB.
In alternative embodiments, the composition is administered in vivo in or in proximity to the diseased, injured or affected tissue.
In alternative embodiments, the neurodegenerative disease or injury, or neurodegenerative condition, is Alzheimer's disease (AD), Parkinson's disease (PD), Huntington's disease (HD), a Polyglutamine (PolyQ) Disease, Amyotrophic lateral sclerosis (ALS), traumatic brain injury (TBI), Chronic traumatic encephalopathy (CTE), a paralysis, a stroke or an ischemic injury.
In alternative embodiments, the invention provides formulations, products of manufacture (e.g., implants, artificial organs or tissues), or kits comprising trans-differentiated or re-programmed cells of the invention.

The details of one or more embodiments of the invention are set forth in the accompanying drawings and the description below. Other features, objects, and advantages of the invention will be apparent from the description and drawings, and from the claims.

All publications, patents, patent applications, and NCBI or PubMed sequences cited herein are hereby expressly incorporated by reference for all purposes.

### DESCRIPTION OF DRAWINGS

The patent or application file contains at least one drawing executed in color. Copies of this patent or patent application publication with color drawing(s) will be provided by the Office upon request and payment of the necessary fee.

FIG. 1(A) illustrates images of the induction of neuronal morphology and the expression of the neuronal marker Tuj1 in multiple cell types in response to depletion of PTB; FIG. 1(B) illustrates images characterizing two cell types (N2A and MEF) with additional neural markers; FIG. 1(C) graphically illustrates the quantification of induced neuronal-like cells derived from N2A and MEFs, wherein the data were based on positive Tuj1 stained cells divided by initial plating cells in response to two separate shPTBs (sh1 and sh2); FIG. 1(D) illustrates images from a time course analysis of neuronal induction on shPTB-treated MEF cells, where MAP2 and NeuN were stained at indicated time points; FIG. 1(E) graphically illustrates the quantified temporal profile of PTB knockdown-induced neurons; all as described in detail in Example 1, below.

FIG. 2(A) illustrates representative traces of whole-cell currents on control shRNA-treated (top) and shPTB-treated (bottom) MEFs; FIG. 2(B) illustrates representative trace of action potentials in response to step current injections on shPTB-induced neurons after co-culturing with rat glial cells; FIG. 2(C) illustrates an image of an shPTB-induced neuron co-cultured with GFP-marked rat glial cells (left panel), where a recording electrode was patched on the shPTB-induced neuron (middle and right panels); FIG. 2(D), FIG. 2(E) and FIG. 2(F) illustrates representative traces of spontaneous postsynaptic currents on shPTB-induced neurons (D), where the cell was held at −70 mV, revealing events of various amplitudes and frequencies, and the insert shows a representative trace of synaptic response, and the insert in FIG. 2(E) illustrates highlights of the remaining GABA current; FIG. 2(G) induction of GABA currents by focal application of 1 mM GABA, which could be blocked by PiTX (red, as labeled “PiTX”); FIG. 2(H) illustrates a representative trace of synaptic currents recorded on shPTB-induced neurons, where Vh is holding potential; all as described in detail in Example 1, below.

FIG. 3(A) graphically illustrates data from an RT-qPCR analysis of a panel of transcription factors and microRNAs in shPTB-treated MEFs; FIG. 3(B) illustrates Western blotting data showing down-regulation of SCP1 in multiple cell types; FIG. 3(C) and FIG. 3(D) illustrate data showing rescue of SCP1 expression in PTB knockdown cells by an shRNA-resistant PTB in HeLa FIG. 3(C) and N2A FIG. 3(D) cells; the data of FIG. 3C is also graphically illustrated; FIG. 3(E) graphically illustrates a time course analysis of neural induction by retinoic acid (RA) on NT2 cells analyzed by RT-qPCR; FIG. 3(F) illustrates images of the induction of neuronal differentiation on MEFs with shRNA against SCP1 or REST; data is also graphically illustrated, where the induction efficiency was calculated based on the number of cells with positive MAP2 and NeuN staining divided by total plating cells; all as described in detail in Example 1, below.

FIG. 4(A) and FIG. 4(B) illustrate PTB-regulated alternative splicing of LSD1 and PHF21A; the CLIP-seq mapped PTB binding events (blue) are shown along with deduced PTB binding peaks (orange, lines) on each gene model; the data is also graphically illustrated in both FIG. 4(B) and FIG. 4(A), where PTB knockdown induced alternative splicing was determined by RT-qPCR in the case of LSD1 and by semi-quantitative RT-PCR in the case of PHF21A; FIG. 4(C) graphically illustrates data showing the relative enrichment of PTB binding in intronic and 3′UTR regions, wherein significant enrichment of PTB binding events is indicated by the p-values in each case; FIG. 4(D) graphically illustrates PTB binding on two REST component genes, showing that multiple PTB binding peaks overlap with validated targeting sites by miR-124 and miR-9; FIG. 4(E) illustrates a gel showing reduced CoREST and HDAC1 proteins (left) and diminished reporter activities (right) in PTB-depleted HeLa cells, and the data is also graphically illustrated in FIG. 4(E); FIG. 4(F) graphically illustrates data showing genome-wide analysis of PTB-regulated RNA stability, where the calculated decay rate was compared in the presence (shCtrl-treated) or absence (shPTB-treated) of PTB; FIG. 4(G) graphically illustrates data showing accelerated SCP1 mRNA decay detected by RT-qPCR in PTB-depleted HeLa cells; FIG. 4(H) graphically illustrates data showing the effect of knocking down PTB (PTB-) or both PTB and Ago2 (PTB-/Ago2-) on the expression of a panel of genes that show PTB and Ago2 binding events in their 3′UTRs, where a gene (UBC) without binding evidence for PTB and Ago2 severed as a negative control; FIG. 4(I) graphically i illustrates data showing re-capture of PTB-dependent regulation with the 3′UTR of individual genes analyzed in H; all as described in detail in Example 1, below.

FIG. 5(A) graphically illustrates data showing the mapped PTB binding events in the 3′UTR of the SCP1 gene (top), where the graphic above the gene model shows the mapped Ago2 binding peaks before (red, see “PTB +” line) and after (black, see “PTB −” line) PTB knockdown in HeLa cells; and the graphic below the gene model indicates multiple predicted microRNA target sites for miR-124 (brown, or first, third, fourth and seventh, lines) and miR-96 (cyan, or second, fifth and sixth, lines), and arrow-highlighted are deduced base-paired regions between the mRNA and individual microRNAs, and also schematically illustrated are the sequence mutations in the 3′UTR of the SCP1 gene that correspond to the sequence on the microRNA targeting sites in the seed region (violet, also labeled “seed M”) or on the PTB binding site (red, also labeled “PTB sites M”) in each case; FIG. 5(B) graphically illustrates data showing the effects on the endogenous SCP1 mRNA by overexpressed miR-96 and its antagomir before and after PTB knockdown; FIG. 5(C) graphically illustrates data showing the blockage of the effect of overexpressed miR-96 and miR-124 by PTB overexpression on the luciferase reporter containing the F1 fragment from the SCP1 3′UTR; FIG. 5(D) graphically illustrates data showing the enhanced effect of overexpressed miR-96 and miR-124 in response to PTB knockdown on the luciferase reporter containing the F1 fragment from the SCP1 3′UTR; FIG. 5(E) graphically illustrates data showing the requirement for the seed region in the miR-96 target site to respond to overexpressed miR-96, where the mutations in the PTB binding site impaired miR-96 targeting (compared lanes 3 and 7), the mutants enhanced the overall effect of miR-96 on the luciferase reporter (compare lanes 3/4 and lanes 7/8); and, FIG. 5(F) graphically illustrates data showing the contribution of individual miR-124 target sites in the SCP1 F1 region to microRNA-mediated down-regulation of the luciferase activity, where the mutations in the seed region of miR-124 targeting sites progressively reduced the response to overexpressed miR-124 (compare lanes 3 to 10), and the mutations in the PTB binding site near the first miR-124 targeting sites enhanced miR-124 mediated down-regulation (compare lanes 4 and 12); all as described in detail in Example 1, below.

FIG. 6(A) graphically illustrates data showing the stabilization of the GNPDA1 transcript in response to PTB and/or Ago2 knockdown in the presence of the transcription inhibitor ActD; FIG. 6(B) schematically illustrates potential microRNA targeting sites near the mapped PTB binding site in the 3′UTR of GNPDA1; FIG. 6(C) graphically illustrates data showing the overexpressed Let-7b suppressed and antagomir Let-7b enhanced the expression of the luciferase reporter containing the 3′UTR of GNPDA1 (lanes 1 to 3), wherein PTB knockdown enhanced the luciferase activity (compared between lanes 1 and 4); FIG. 6(D) illustrates a Western blot showing antagomir Let-7b, miR-196a and miR-181b increased GNPDA1 protein in the presence, but not absence, of PTB in transfected HeLa cells, and the protein levels were quantified with the SD shown in the bottom; FIG. 6(E) and FIG. 6 (F) illustrate the mapping of the secondary structure in the 3′UTR of GNPDA1, where the gel illustrated in FIG. 6(E) shows individual G residues labeled on the left (with red, or residues 52G, 42G, 32G, 19G, 16G, and 11G) indicating several key positions in the deduced secondary structure (E), as illustrated in the gel of FIG. 6(E), where red (the T1-PTB+ lane) and blue (the V1-PTB+ lane) arrows respectively indicate PTB enhanced and suppressed cleavages in the deduced stem-loop region, and the quantified fold-changes at key positions are indicated in the box inserted in the panel of FIG. 6(F); FIG. 6(G) and FIG. 6(H) illustrates data showing increased single-strandness of RNA in the presence of increasing amounts of PTB detected by in-line probing, as illustrated in the gel of FIG. 6(G), and as schematically illustrated in FIG. 6(H), a proposed model indicates PTB-mediated opening of the stem-loop that facilitates microRNA targeting; all as described in detail in Example 1, below.

FIG. 7(A) illustrates a Western blot showing CLIP signals detected with anti-Ago2 before and after PTB knockdown; FIG. 7(B) graphically illustrates a data comparison between the two Ago2 CLIP-seq datasets in 1 kb windows across the human genome before and after PTB depletion; FIG. 7(C) graphically illustrates a pie chart showing the genomic distribution of Ago2 binding events before (left) and after (right) PTB knockdown, showing prevalent Ago2 binding in the 3′UTR region; FIG. 7(D) and FIG. 7(E) graphically illustrates data showing Ago2 binding in the 3′UTR of PTB unbound FIG. 7(D) and bound FIG. 7(E) targets before (red, lower line) and after (blue, upper line) PTB knockdown; FIG. 7(F) graphically illustrates data of an induction of significant Ago2 binding on and near the PTB binding sites; FIG. 7(G) graphically illustrates data showing the functional correlation between PTB/microRNA interplay and gene expression, where the genes with induced and repressed expression are plotted in a cumulative fashion; and, FIG. 7(H) schematically illustrates a model for the PTB-regulated miR124-REST loop; all as described in detail in Example 1, below

FIG. 8 illustrates Table 1, a list of primers for RT-PCR and construction of luciferase reporters, as described in detail in Example 1, below.

FIG. 9, or FIG. S1, illustrates: FIG. 9(A) (left) illustrates a Western blotting analysis showing the induction of nPTB as well as a neuronal marker MAP2 in PTB knockdown HeLa cells, FIG. 9A(A) (right) illustrates HeLa cells depleted of PTB exhibited neurite outgrowth; FIG. 9(B) illustrates Western blotting analysis showing efficient knockdown of PTB with two different shPTBs in MEFs (upper gel) and N2A (lower gel) cells; FIG. 9(C) illustrates images of stained cells showing evidence for the lack of contaminating neurons or neural crest cells based on immunostaining for a large number of neural markers as shown, where each antibody was individually validated using appropriate positive controls, including neural progenitors isolated from E14.5 mouse brain, which were stained for P75, Pax3, Pax7, NKX2.2, Brn2 and Olig1; shPTB-induced MEFs for Tuj1; human fetal retinal progenitor for Sox2 and Pax6; and mouse muller glial cells for GFAP; FIG. 9(D) illustrates a gel analysis showing evidence for the lack of contaminating neurons or neural crest cells based on RT-PCR analysis against a large panel of neural specific genes; FIG. 9(E) illustrates images of stained cells showing induction of neuronal differentiation in both N2A and MEFs with two different shRNAs against PTB (PTB#1 and PTB#2) and rescue of the phenotype with specific shRNA-resistant, FLAG tagged PTB expression units (FLAG-M1 and FLAG-M2) that contain synonymous mutants in each shPTB targeting site; as described in detail in Example 1, below.

FIG. 10, or FIG. S2, illustrates: FIG. 10(A) illustrates representative traces of whole-cell currents in a voltage-clamp mode and depolarization-induced single action potential on induced neuronal like cells derived from N2A cells; FIG. 10(B) illustrates cell images in time sequence (second) where rapid Ca⁺⁺ influx was measured using Fluo-5-AM in response to membrane depolarization on shPTB-induced neuronal like cells from N2A cells; FIG. 10(C) illustrates cell images of rapid Ca⁺⁺ influx as measured using Fluo-5-AM in response to membrane depolarization on shPTB-induced neuronal like cells from MEFs; as described in detail in Example 1, below.

FIG. 11, or FIG. S3, illustrates: FIG. 11(A) graphically illustrates an RNA-seq analysis of gene expression in response to PTB knockdown in HeLa cells; significantly up- and down-regulated genes labeled red and blue, respectively, with green dots representing those that have neuronal-related functions documented in literature; FIG. 11(B) graphically illustrates an RT-qPCR validation of a panel of genes that were altered to different degrees (blue) as well several housekeeping genes (purple) in response to PTB knockdown in HeLa cells, and the data were plotted against the RNA-seq results, and red indicates three cases where the qPCR results were not consistent with the RNA-seq results; FIG. 11(C) graphically illustrates Gene Ontology (GO) analysis of PTB-regulated genes, where the top enriched GO terms (−log₂(p)>10) are highlighted for both up-regulated (red, upper graph) and down-regulated (blue, lower graph) genes that are related to neuronal functions; FIG. 11(D) graphically illustrates data showing confirmation of REST binding (right bar on graph) on a panel of shPTB-induced genes by ChIP-qPCR on MEFs, where IgG (left bar) was test as a control; FIG. 11(E) graphically illustrates data showing induction of multiple neuronal specific genes in MEFs treated with REST RNAi; FIG. 11(F) in chart form illustrates data showing a comparison between PTB-regulated splicing events previously reported (Makeyev et al., 2007) and their splicing changes in PTB knockdown cells determined by RNA-seq in this study; FIG. 11(G) schematically illustrates a REST splicing event, where inclusion of the neuronal exon (N) will result in the production of the REST4 isoform, which encodes a truncated, non-functional REST protein; as described in detail in Example 1, below.

FIG. 12, or FIG. S4, illustrates: FIG. 12(A) in chart form illustrates data from previously reported cases of PTB-regulated RNA stability that contain predicted microRNA targeting sites on the mapped PTB binding sites; FIG. 12(B) schematically illustrates an MS2 tethering approach, where a phage RNA binding motif (MS2) was introduced to a 3′UTR of a luciferase reporter, where a mutant MS2 motif containing a point mutation known to disrupt binding by the MS2 RNA binding domain served as a negative control; FIG. 12(C) illustrates a Western blot of PTB-MS2 fusion protein showing levels of the PTB-MS2 fusion protein expressed in HeLa cells co-transfected with wild type and mutant reporters; FIG. 12(D) illustrates a Western blot of PTB-MS2 fusion protein showing a lack of influence of overexpressed PTB-MS2 fusion protein on the luciferase activity; as described in detail in Example 1, below.

FIG. 13, or FIG. S5, illustrates: FIGS. 13(A), (B) and (C) graphically illustrates data from luciferase reporter assays on the entire SCP1 3′UTR (FIG. 13(A)), the F2 fragment from the SCP1 3′UTR (FIG. 13 (B)) and the F3 fragment from the SCP1 3′UTR (FIG. 13 (C)); FIG. 13(D) graphically illustrates data from a PTB-induced switch in alternative polyadenylation, alternative polyadenylation events induced by PTB knockdown were measured; FIG. 13(E) graphically illustrates data from a statistical analysis based on two-sided Kolmogorov-Smirnov test that indicates that PTB knockdown caused little global changes in alternative polyadenylation; as described in detail in Example 1, below.

FIG. 14, or FIG. S6, illustrates: FIG. 14(A) illustrates a gel shift analysis of PTB binding on the mapped PTB binding site near the microRNA regulatory element (MRE) in the 3′UTR of the GNPDA1 gene (upper gel), compared to a gel shift analysis of PTB binding in an HBV genome (lower gel); FIG. 14(B) graphically illustrates (upper illustration) the 3′UTR of the GNPDA1 gene as cloned into a luciferase reporter, where reporter activity was increased in response to double knockdown of PTB and nPTB in NT2 cells without (compare between lanes 3 and 4) or with Let-7b overexpression (compare between lanes 7 and 8), and where Western blotting validated the knockdown efficiency of PTB and nPTB (bottom gel illustration); as described in detail in Example 1, below.

FIG. 15, or FIG. S7, illustrates: FIG. 15(A) graphically illustrates a comparison of genes in group 2 (blue line, with binding evidence for Ago2, but not PTB) with genes in group 4 (green line) that showed both Ago2 and PTB binding, but little overlap between their binding events, and with genes in group 5 (purple line) that exhibited overlapped binding events between Ago2 and PTB (at least one pair of peaks separated by <10 nt); and FIG. 15(B) graphically illustrates a comparison of genes in group 3 (coffee-colored line that showed binding evidence for PTB, but not Ago2) with genes in group 4 and 5; all as described in detail in Example 1, below.

FIG. 16 illustrates data demonstrating that sequential PTB knockdown followed by nPTB knockout efficiently converted human fibroblasts to neurons with mature neuronal markers, such as MAP2, RFP, TUJ1: FIG. 16A schematically illustrates the protocol (including culture media used) and time line of the experiment; and FIG. 16B illustrates cellular images stained over time for the expression of the mature neuronal markers MAP2, RFP, TUJ1; as described in detail in Example 1, below.

Like reference symbols in the various drawings indicate like elements.

### DETAILED DESCRIPTION

The invention provides compositions and in vivo, ex vivo and in vitro methods for trans-differentiation of, re-differentiating or re-programming mammalian cells to functional neurons. In alternative embodiments, the invention provides compositions capable of inactivating RNA polypyrimidine tract binding protein (PTB) for de-differentiating, re-differentiating or re-programming mammalian cells. The invention also provides compositions and methods for direct reprogramming, or trans-differentiation, of a first differentiated phenotype of a cell to a second differentiated phenotype, or to a functioning neuron.

This invention for the first time demonstrates that inactivation of a single RNA polypyrimidine tract binding protein (PTB) is sufficient to induce the expression of a specific set of transcription factors, which act together to trigger trans-differentiation of diverse cell types into functional neurons. The inventors identified a key gene that acts to regulate these factors. The invention demonstrates that PTB, which is naturally down regulated during brain development, is involved in regulating RNA metabolism at both the transcript splicing and microRNA (miRNA) levels. In alternative embodiments, the invention provides compositions and methods for engineering non-neuronal cells into neurons.

The inventors found that a single RNA binding protein PTB, which is naturally down regulated during brain development, is involved in regulating RNA metabolism at both the splicing and microRNA levels. The function of PTB in regulating microRNA targeting in the human genome was first demonstrated in this study. These functions cause a series of molecular switches, a most important one being the inactivation of the RE1-Silencing Transcription factor (REST; also known as Neuron-Restrictive Silencer Factor, or NRSF) complex. This leads to the induction of a series of neuronal specific genes in non-neuronal cells. In the presence of other neural trophic factors, the morphologically transformed cells become functional neurons.

The inventors identified a key gene, the PTB gene, that acts to regulate transcription factors controlling trans-differentiation of diverse cell types into functional neurons. As a result, the invention for the first time demonstrates that altered expression of the PTB gene is sufficient to induce all morphological and functional changes towards the neural lineage. In one embodiment, methods of the invention inactivates the PTB gene to regulate transcription factors to trans-differentiate diverse cell types into functional neurons; this embodiment inactivates a gene, as compared to overexpressing a number of genes together, to switch a cell fate, e.g., into functional neurons.

In alternative embodiments, the invention provides compositions and methods for engineering non-neuronal cells in vivo or ex vivo into neurons in the central nervous system (CNS), e.g., the brain or spinal cord, to treat an injury, condition or disease, e.g., a neurodegenerative disease, a spinal injury, a paralysis due to an injury or disease, and the like.

The present invention demonstrates that regulated PTB expression is able to induce massive reprogramming at both the splicing and microRNA levels to drive the cell fate decision towards the neuronal lineage. Thus, the invention provides compositions and methods for manipulating, e.g., trans-differentiating or re-programming, mammalian cell phenotypes, e.g., human or animal cell phenotypes, comprising use of compositions or compounds, e.g., proteins (e.g., antibodies, aptamers), nucleic acids (e.g., antisense or miRNA), small molecules and the like, to inactivation of an RE1-Silencing Transcription factor (REST; also known as Neuron-Restrictive Silencer Factor, or NRSF) complex or inactivate the Polypyrimidine Tract Binding protein (PTB) gene.

### Antibodies, Therapeutic and Humanized Antibodies

In alternative embodiments, the invention provides antibodies that specifically bind to and inhibit: an RE1-Silencing Transcription factor (REST; also known as Neuron-Restrictive Silencer Factor, or NRSF) complex, or, a Polypyrimidine Tract Binding protein (PTB) gene or protein.

In alternative embodiments, the invention uses isolated, synthetic or recombinant antibodies that specifically bind to and inhibit or activate a PTB gene or protein.

In alternative aspects, an antibody for practicing the invention can comprise a peptide or polypeptide derived from, modeled after or substantially encoded by an immunoglobulin gene or immunoglobulin genes, or fragments thereof, capable of specifically binding an antigen or epitope, see, e.g. Fundamental Immunology, Third Edition, W. E. Paul, ed., Raven Press, N.Y. (1993); Wilson (1994) J. Immunol. Methods 175:267-273; Yarmush (1992) J. Biochem. Biophys. Methods 25:85-97. In alternative aspects, an antibody for practicing the invention includes antigen-binding portions, i.e., “antigen binding sites,” (e.g., fragments, subsequences, complementarity determining regions (CDRs)) that retain capacity to bind antigen, including (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Single chain antibodies are also included by reference in the term “antibody.”

Methods of immunization, producing and isolating antibodies (polyclonal and monoclonal) are known to those of skill in the art and described in the scientific and patent literature, see, e.g., Coligan, CURRENT PROTOCOLS IN IMMUNOLOGY, Wiley/Greene, NY (1991); Stites (eds.) BASIC AND CLINICAL IMMUNOLOGY (7th ed.) Lange Medical Publications, Los Altos, Calif. (“Stites”); Goding, MONOCLONAL ANTIBODIES: PRINCIPLES AND PRACTICE (2d ed.) Academic Press, New York, N.Y. (1986); Kohler (1975) Nature 256:495; Harlow (1988) ANTIBODIES, A LABORATORY MANUAL, Cold Spring Harbor Publications, New York. Antibodies also can be generated in vitro, e.g., using recombinant antibody binding site expressing phage display libraries, in addition to the traditional in vivo methods using animals. See, e.g., Hoogenboom (1997) Trends Biotechnol. 15:62-70; Katz (1997) Annu. Rev. Biophys. Biomol. Struct. 26:27-45.

In alternative embodiments, the invention uses “humanized” antibodies, including forms of non-human (e.g., murine) antibodies that are chimeric antibodies comprising minimal sequence (e.g., the antigen binding fragment) derived from non-human immunoglobulin. In alternative embodiments, humanized antibodies are human immunoglobulins in which residues from a hypervariable region (HVR) of a recipient (e.g., a human antibody sequence) are replaced by residues from a hypervariable region (HVR) of a non-human species (donor antibody) such as mouse, rat, rabbit or nonhuman primate having the desired specificity, affinity, and capacity. In alternative embodiments, framework region (FR) residues of the human immunoglobulin are replaced by corresponding non-human residues to improve antigen binding affinity.

In alternative embodiments, humanized antibodies may comprise residues that are not found in the recipient antibody or the donor antibody. These modifications may be made to improve antibody affinity or functional activity. In alternative embodiments, the humanized antibody can comprise substantially all of at least one, and typically two, variable domains, in which all or substantially all of the hypervariable regions correspond to those of a non-human immunoglobulin and all or substantially all of Ab framework regions are those of a human immunoglobulin sequence.

In alternative embodiments, a humanized antibody used to practice this invention can comprise at least a portion of an immunoglobulin constant region (Fc), typically that of or derived from a human immunoglobulin.

However, in alternative embodiments, completely human antibodies also can be used to practice this invention, including human antibodies comprising amino acid sequence which corresponds to that of an antibody produced by a human. This definition of a human antibody specifically excludes a humanized antibody comprising non-human antigen binding residues.

In alternative embodiments, antibodies used to practice this invention comprise “affinity matured” antibodies, e.g., antibodies comprising with one or more alterations in one or more hypervariable regions which result in an improvement in the affinity of the antibody for antigen; e.g., a targeted transcriptional activating factor, compared to a parent antibody which does not possess those alteration(s). In alternative embodiments, antibodies used to practice this invention are matured antibodies having nanomolar or even picomolar affinities for the target antigen, e.g., a targeted transcriptional activating factor. Affinity matured antibodies can be produced by procedures known in the art.

### Generating and Manipulating Nucleic Acids

In alternative aspects, composition and methods of the invention comprise use of nucleic acids for inactivating an RE1-Silencing Transcription factor (REST; also known as Neuron-Restrictive Silencer Factor, or NRSF) complex, or, inactivating a Polypyrimidine Tract Binding protein (PTB) gene or protein.

In alternative embodiments, nucleic acids of the invention are made, isolated and/or manipulated by, e.g., cloning and expression of cDNA libraries, amplification of message or genomic DNA by PCR, and the like.

The nucleic acids used to practice this invention, whether RNA, iRNA, antisense nucleic acid, cDNA, genomic DNA, vectors, viruses or hybrids thereof, can be isolated from a variety of sources, genetically engineered, amplified, and/or expressed/ generated recombinantly. Any recombinant expression system can be used, including e.g. bacterial, fungal, mammalian, yeast, insect or plant cell expression systems.

Alternatively, nucleic acids used to practice this invention can be synthesized in vitro by well-known chemical synthesis techniques, as described in, e.g., Adams (1983) J. Am. Chem. Soc. 105:661; Belousov (1997) Nucleic Acids Res. 25:3440-3444; Frenkel (1995) Free Radic. Biol. Med. 19:373-380; Blommers (1994) Biochemistry 33:7886-7896; Narang (1979) Meth. Enzymol. 68:90; Brown (1979) Meth. Enzymol. 68:109; Beaucage (1981) Tetra. Lett. 22:1859; U.S. Pat. No. 4,458,066.

Techniques for the manipulation of nucleic acids used to practice this invention, such as, e.g., subcloning, labeling probes (e.g., random-primer labeling using Klenow polymerase, nick translation, amplification), sequencing, hybridization and the like are well described in the scientific and patent literature, see, e.g., Sambrook, ed., MOLECULAR CLONING: A LABORATORY MANUAL (2ND ED.), Vols. 1-3, Cold Spring Harbor Laboratory, (1989); CURRENT PROTOCOLS IN MOLECULAR BIOLOGY, Ausubel, ed. John Wiley & Sons, Inc., New York (1997); LABORATORY TECHNIQUES IN BIOCHEMISTRY AND MOLECULAR BIOLOGY: HYBRIDIZATION WITH NUCLEIC ACID PROBES, Part I. Theory and Nucleic Acid Preparation, Tijssen, ed. Elsevier, N.Y. (1993).

Another useful means of obtaining and manipulating nucleic acids used to practice the methods of the invention is to clone from genomic samples, and, if desired, screen and re-clone inserts isolated or amplified from, e.g., genomic clones or cDNA clones. Sources of nucleic acid used in the methods of the invention include genomic or cDNA libraries contained in, e.g., mammalian artificial chromosomes (MACs), see, e.g., U.S. Pat. Nos. 5,721,118; 6,025,155; human artificial chromosomes, see, e.g., Rosenfeld (1997) Nat. Genet. 15:333-335; yeast artificial chromosomes (YAC); bacterial artificial chromosomes (BAC); P1 artificial chromosomes, see, e.g., Woon (1998) Genomics 50:306-316; P1-derived vectors (PACs), see, e.g., Kern (1997) Biotechniques 23:120-124; cosmids, recombinant viruses, phages or plasmids.

Nucleic acids or nucleic acid sequences used to practice this invention can be an oligonucleotide, nucleotide, polynucleotide, or to a fragment of any of these, to DNA or RNA of genomic or synthetic origin which may be single-stranded or double-stranded and may represent a sense or antisense strand, to peptide nucleic acid (PNA), or to any DNA-like or RNA-like material, natural or synthetic in origin. Compounds use to practice this invention include “nucleic acids” or “nucleic acid sequences” including oligonucleotide, nucleotide, polynucleotide, or any fragment of any of these; and include DNA or RNA (e.g., mRNA, rRNA, tRNA, iRNA) of genomic or synthetic origin which may be single-stranded or double-stranded; and can be a sense or antisense strand, or a peptide nucleic acid (PNA), or any DNA-like or RNA-like material, natural or synthetic in origin, including, e.g., iRNA, ribonucleoproteins (e.g., e.g., double stranded iRNAs, e.g., iRNPs). Compounds use to practice this invention include nucleic acids, i.e., oligonucleotides, containing known analogues of natural nucleotides. Compounds use to practice this invention include nucleic-acid-like structures with synthetic backbones, see e.g., Mata (1997) Toxicol. Appl. Pharmacol. 144:189-197; Strauss-Soukup (1997) Biochemistry 36:8692-8698; Samstag (1996) Antisense Nucleic Acid Drug Dev 6:153-156. Compounds use to practice this invention include “oligonucleotides” including a single stranded polydeoxynucleotide or two complementary polydeoxynucleotide strands that may be chemically synthesized. Compounds use to practice this invention include synthetic oligonucleotides having no 5′ phosphate, and thus will not ligate to another oligonucleotide without adding a phosphate with an ATP in the presence of a kinase. A synthetic oligonucleotide can ligate to a fragment that has not been dephosphorylated.

### Antisense Inhibitory Nucleic Acid Molecules

In alternative embodiments, the invention provides antisense or otherwise inhibitory nucleic acid molecules capable of decreasing or inhibiting expression of: an RE1-Silencing Transcription factor (REST; also known as Neuron-Restrictive Silencer Factor, or NRSF) complex; a Polypyrimidine Tract Binding protein (PTB) gene or protein, e.g., a neuronal-specific miR-124; and/or a nPTB. In alternative embodiments, methods of the invention comprise use of molecules that can generate a PTB and a nPTB knockdown, or abrogation or significant decrease in PTB and nPTB expression. In alternative embodiments, methods of the invention comprise use of these molecules to sequentially knockout first PTB, then nPTB, thus efficiently converting a human cell (e.g., a fibroblast) to a functional neuronal cell with mature neuronal marks, such as MAP2. It was demonstrated that nPTB has to be knocked down 4 days or later to achieve this phenotype. Accordingly, this exemplary embodiment provides methods for converting non-neuronal human cells to functional neurons for regenerative medicine.

The sequences of PTB and nPTB are known (see e.g., Romanelli et al. (2005) Gene, August 15:356:11-8; Robinson et al., PLoS One. 2008 Mar. 12; 3(3):e1801. doi: 10.1371/journal.pone.0001801; Makeyev et al., Mol. Cell (2007) August 3; 27(3):435-48); thus, one of skill in the art can design and construct antisense, miRNA, siRNA molecules and the like to modulate, e.g., to decrease or inhibit, the expression of PTB and/or nPTB; to practice the methods of this invention.

Naturally occurring or synthetic nucleic acids can be used as antisense oligonucleotides. The antisense oligonucleotides can be of any length; for example, in alternative aspects, the antisense oligonucleotides are between about 5 to 100, about 10 to 80, about 15 to 60, about 18 to 40. The optimal length can be determined by routine screening. The antisense oligonucleotides can be present at any concentration. The optimal concentration can be determined by routine screening. A wide variety of synthetic, non-naturally occurring nucleotide and nucleic acid analogues are known which can address this potential problem. For example, peptide nucleic acids (PNAs) containing non-ionic backbones, such as N-(2-aminoethyl) glycine units can be used. Antisense oligonucleotides having phosphorothioate linkages can also be used, as described in WO 97/03211; WO 96/39154; Mata (1997) Toxicol Appl Pharmacol 144:189-197; Antisense Therapeutics, ed. Agrawal (Humana Press, Totowa, N.J., 1996). Antisense oligonucleotides having synthetic DNA backbone analogues provided by the invention can also include phosphoro-dithioate, methylphosphonate, phosphoramidate, alkyl phosphotriester, sulfamate, 3′-thioacetal, methylene(methylimino), 3′-N-carbamate, and morpholino carbamate nucleic acids.

RNA Interference (RNAi)

In alternative embodiments, the invention uses RNAi inhibitory nucleic acid molecules capable of decreasing or inhibiting expression of: an RE1-Silencing Transcription factor (REST; also known as Neuron-Restrictive Silencer Factor, or NRSF) complex, or, a Polypyrimidine Tract Binding protein (PTB) or nPTB gene, message or protein.

In one aspect, the RNAi molecule comprises a double-stranded RNA (dsRNA) molecule. The RNAi molecule can comprise a double-stranded RNA (dsRNA) molecule, e.g., siRNA, miRNA (microRNA) and/or short hairpin RNA (shRNA) molecules. For example, in one embodiment, the invention uses inhibitory, e.g., siRNA, miRNA or shRNA, nucleic acids that inhibit or suppress the activity of a tumor suppressor gene retinoblastoma-1 (RB1) and/or a p53 tumor suppressor gene (TP53).

In alternative aspects, the RNAi is about 11, 12, 13, 14, 15, 16, 17, 18, 19, 20, 21, 22, 23, 24, 25 or more duplex nucleotides in length. While the invention is not limited by any particular mechanism of action, the RNAi can enter a cell and cause the degradation of a single-stranded RNA (ssRNA) of similar or identical sequences, including endogenous mRNAs. When a cell is exposed to double-stranded RNA (dsRNA), mRNA from the homologous gene is selectively degraded by a process called RNA interference (RNAi). A possible basic mechanism behind RNAi, e.g., siRNA for inhibiting transcription and/or miRNA to inhibit translation, is the breaking of a double-stranded RNA (dsRNA) matching a specific gene sequence into short pieces called short interfering RNA, which trigger the degradation of mRNA that matches its sequence. In one aspect, the RNAi's of the invention are used in gene-silencing therapeutics, e.g., to silence one or a set of transcription factors responsible for maintaining the differentiated phenotype of the differentiated cell; see, e.g., Shuey (2002) Drug Discov. Today 7:1040-1046. In one aspect, the invention provides methods to selectively degrade an RNA using the RNAi's of the invention. In one aspect, the RNAi molecules of the invention can be used to generate a loss-of-function mutation in a cell. These processes may be practiced in vitro, ex vivo or in vivo.

In one aspect, intracellular introduction of the RNAi (e.g., miRNA or siRNA) is by internalization of a target cell specific ligand bonded to an RNA binding protein comprising an RNAi (e.g., microRNA) is adsorbed. The ligand can be specific to a unique target cell surface antigen. The ligand can be spontaneously internalized after binding to the cell surface antigen. If the unique cell surface antigen is not naturally internalized after binding to its ligand, internalization can be promoted by the incorporation of an arginine-rich peptide, or other membrane permeable peptide, into the structure of the ligand or RNA binding protein or attachment of such a peptide to the ligand or RNA binding protein. See, e.g., U.S. Patent App. Pub. Nos. 20060030003; 20060025361; 20060019286; 20060019258. In one aspect, the invention provides lipid-based formulations for delivering, e.g., introducing nucleic acids of the invention as nucleic acid-lipid particles comprising an RNAi molecule to a cell, see e.g., U.S. Patent App. Pub. No. 20060008910.

Methods for making and using RNAi molecules, e.g., siRNA and/or miRNA, for selectively degrade RNA are well known in the art, see, e.g., U.S. Pat. Nos. 6,506,559; 6,511,824; 6,515,109; 6,489,127.

Methods for making expression constructs, e.g., vectors or plasmids, from which an inhibitory polynucleotide (e.g., a duplex siRNA of the invention) is transcribed are well known and routine. A regulatory region (e.g., promoter, enhancer, silencer, splice donor, acceptor, etc.) can be used to transcribe an RNA strand or RNA strands of an inhibitory polynucleotide from an expression construct. When making a duplex siRNA inhibitory molecule, the sense and antisense strands of the targeted portion of the targeted IRES can be transcribed as two separate RNA strands that will anneal together, or as a single RNA strand that will form a hairpin loop and anneal with itself. For example, a construct targeting a portion of a gene, e.g., an NADPH oxidase enzyme coding sequence or transcriptional activation sequence, is inserted between two promoters (e.g., mammalian, viral, human, tissue specific, constitutive or other type of promoter) such that transcription occurs bidirectionally and will result in complementary RNA strands that may subsequently anneal to form an inhibitory siRNA of the invention.

Alternatively, a targeted portion of a gene, coding sequence, promoter or transcript can be designed as a first and second antisense binding region together on a single expression vector; for example, comprising a first coding region of a targeted gene in sense orientation relative to its controlling promoter, and wherein the second coding region of the gene is in antisense orientation relative to its controlling promoter. If transcription of the sense and antisense coding regions of the targeted portion of the targeted gene occurs from two separate promoters, the result may be two separate RNA strands that may subsequently anneal to form a gene-inhibitory siRNA used to practice this invention.

In another aspect, transcription of the sense and antisense targeted portion of the targeted gene is controlled by a single promoter, and the resulting transcript will be a single hairpin RNA strand that is self-complementary, i.e., forms a duplex by folding back on itself to create a gene-inhibitory siRNA molecule. In this configuration, a spacer, e.g., of nucleotides, between the sense and antisense coding regions of the targeted portion of the targeted gene can improve the ability of the single strand RNA to form a hairpin loop, wherein the hairpin loop comprises the spacer. In one embodiment, the spacer comprises a length of nucleotides of between about 5 to 50 nucleotides. In one aspect, the sense and antisense coding regions of the siRNA can each be on a separate expression vector and under the control of its own promoter.

Inhibitory Ribozymes

In alternative embodiments, the invention uses ribozymes capable of decreasing or inhibiting expression of: an RE1-Silencing Transcription factor (REST; also known as Neuron-Restrictive Silencer Factor, or NRSF) complex, or, a Polypyrimidine Tract Binding protein (PTB) or nPTB gene, message or protein.

These ribozymes can inhibit a gene's activity by, e.g., targeting a genomic DNA or an mRNA (a message, a transcript). Strategies for designing ribozymes and selecting a gene-specific antisense sequence for targeting are well described in the scientific and patent literature, and the skilled artisan can design such ribozymes using the novel reagents of the invention. Ribozymes act by binding to a target RNA through the target RNA binding portion of a ribozyme which is held in close proximity to an enzymatic portion of the RNA that cleaves the target RNA. Thus, the ribozyme recognizes and binds a target RNA through complementary base-pairing, and once bound to the correct site, acts enzymatically to cleave and inactivate the target RNA. Cleavage of a target RNA in such a manner will destroy its ability to direct synthesis of an encoded protein if the cleavage occurs in the coding sequence. After a ribozyme has bound and cleaved its RNA target, it can be released from that RNA to bind and cleave new targets repeatedly.

### Kits and Instructions

The invention provides kits comprising compositions and methods of the invention, including instructions for use thereof. As such, kits, cells, vectors and the like can also be provided.

For example, in alternative embodiments, the invention provides kits comprising compositions capable of decreasing or inhibiting expression of: an RE1-Silencing Transcription factor (REST; also known as Neuron-Restrictive Silencer Factor, or NRSF) complex, or, a Polypyrimidine Tract Binding protein (PTB) or nPTB gene, message or protein, for e.g., trans-differentiating or re-programming a mammalian cell. In alternative embodiments, the kits comprise instruction for practicing methods of the invention.

### Formulations

In alternative embodiments, the invention provides compositions and formulations for use in in vitro, ex vivo or in vivo methods of the invention for trans-differentiating, re-differentiating or re-programming a mammalian cell to a neuronal cell. In alternative embodiments, these compositions comprise a plurality of (a set of) proteins and/or nucleic acids formulated for these purposes (e.g., to decrease or inhibit expression of a PTB and nPTB gene, message or protein), e.g., formulated in a buffer, in a saline solution, in a powder, an emulsion, in a vesicle, in a liposome, in a nanoparticle, in a nanolipoparticle and the like.

In alternative embodiments, the compositions can be formulated in any way and can be applied in a variety of concentrations and forms depending on the desired in vitro, ex vivo or in vivo conditions, a desired in vitro, ex vivo or in vivo method of administration and the like. Details on techniques for in vitro, ex vivo or in vivo formulations and administrations are well described in the scientific and patent literature.

Formulations and/or carriers used to practice this invention can be in forms such as tablets, pills, powders, capsules, liquids, gels, syrups, slurries, suspensions, etc., suitable for in vitro, ex vivo or in vivo applications.

Compositions used to practice this invention can be in admixture with an aqueous and/or buffer solution or as an aqueous and/or buffered suspension, e.g., including a suspending agent, such as sodium carboxymethylcellulose, methylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate. Formulations can be adjusted for osmolarity, e.g., by use of an appropriate buffer.

In practicing this invention, the compounds (e.g., formulations) of the invention can comprise a solution of nucleic acids (e.g., a neuronal-specific miR-124) or other nucleic acids dissolved in a pharmaceutically acceptable carrier, e.g., acceptable vehicles and solvents that can be employed include water and Ringer's solution, an isotonic sodium chloride. In addition, sterile fixed oils can be employed as a solvent or suspending medium. For this purpose any fixed oil can be employed including synthetic mono- or diglycerides, or fatty acids such as oleic acid. In one embodiment, solutions and formulations used to practice the invention are sterile and can be manufactured to be generally free of undesirable matter. In one embodiment, these solutions and formulations are sterilized by conventional, well known sterilization techniques.

The solutions and formulations used to practice the invention can comprise auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of active agent (e.g., a neuronal-specific miR-124) in these formulations can vary widely, and can be selected primarily based on fluid volumes, viscosities and the like, in accordance with the particular mode of in vitro, ex vivo or in vivo administration selected and the desired results, e.g., for trans-differentiating or re-programming a mammalian cell.

The solutions and formulations used to practice the invention can be lyophilized; for example, the invention provides a stable lyophilized formulation comprising a neuronal-specific miR-124. In one aspect, this formulation is made by lyophilizing a solution comprising an active agent used to practice the invention and a bulking agent, e.g., mannitol, trehalose, raffinose, and sucrose or mixtures thereof. A process for preparing a stable lyophilized formulation can include lyophilizing a solution about 2.5 mg/mL protein, about 15 mg/mL sucrose, about 19 mg/mL NaCl, and a sodium citrate buffer having a pH greater than 5.5 but less than 6.5. See, e.g., U.S. patent app. no. 20040028670.

The compositions and formulations of the invention can be delivered by the use of liposomes (see also discussion, below). By using liposomes, particularly where the liposome surface carries ligands specific for target cells, or are otherwise preferentially directed to a specific tissue or organ type, one can focus the delivery of the active agent into a target cells in an in vitro, ex vivo or in vivo application.

### Nanoparticles, Nanolipoparticles and Liposomes

The invention also provides nanoparticles, nanolipoparticles, vesicles and liposomal membranes comprising compounds used to practice methods of this invention (e.g., compounds to decrease or inhibit expression of a PTB or nPTB gene, message or protein), e.g., to deliver compositions of the invention to mammalian cells in vitro, ex vivo or in vivo. In alternative embodiments, these compositions are designed to target specific molecules, including biologic molecules, such as polypeptides, including cell surface polypeptides, e.g., for targeting a desired cell type, e.g., a mammalian cell targeted for trans-differentiation or re-programming.

The invention provides multilayered liposomes comprising compounds used to practice this invention, e.g., as described in Park, et al., U.S. Pat. Pub. No. 20070082042. The multilayered liposomes can be prepared using a mixture of oil-phase components comprising squalane, sterols, ceramides, neutral lipids or oils, fatty acids and lecithins, to about 200 to 5000 nm in particle size, to entrap a composition used to practice this invention (e.g., a neuronal-specific miR-124).

Liposomes can be made using any method, e.g., as described in Park, et al., U.S. Pat. Pub. No. 20070042031, including method of producing a liposome by encapsulating an active agent (e.g., a composition used to practice this invention, e.g., a neuronal-specific miR-124), the method comprising providing an aqueous solution in a first reservoir; providing an organic lipid solution in a second reservoir, and then mixing the aqueous solution with the organic lipid solution in a first mixing region to produce a liposome solution, where the organic lipid solution mixes with the aqueous solution to substantially instantaneously produce a liposome encapsulating the active agent; and immediately then mixing the liposome solution with a buffer solution to produce a diluted liposome solution.

In one embodiment, liposome compositions used to practice this invention comprise a substituted ammonium and/or polyanions, e.g., for targeting delivery of a composition used to practice this invention, e.g., a neuronal-specific miR-124, to a desired cell type, as described e.g., in U.S. Pat. Pub. No. 20070110798.

The invention also provides nanoparticles comprising a composition used to practice this invention, e.g., a neuronal-specific miR-124, in the form of active agent-containing nanoparticles (e.g., a secondary nanoparticle), as described, e.g., in U.S. Pat. Pub. No. 20070077286. In one embodiment, the invention provides nanoparticles comprising a fat-soluble active agent of this invention or a fat-solubilized water-soluble active agent to act with a bivalent or trivalent metal salt.

In one embodiment, solid lipid suspensions can be used to formulate and to deliver a composition used to practice this invention, e.g., a neuronal-specific miR-124, to mammalian cells in vitro, ex vivo or in vivo, as described, e.g., in U.S. Pat. Pub. No. 20050136121.

### Peptide Delivery Vehicles

In alternative embodiments, any delivery vehicle can be used to practice the methods or compositions of this invention, e.g., to deliver a composition used to practice this invention (e.g., compounds to decrease or inhibit expression of a PTB or nPTB gene, message or protein), e.g., a neuronal-specific miR-124, to mammalian cells in vitro, ex vivo or in vivo. For example, delivery vehicles comprising polycations, cationic polymers and/or cationic peptides, such as polyethyleneimine derivatives, can be used e.g. as described, e.g., in U.S. Pat. Pub. No. 20060083737.

In one embodiment, a dried polypeptide-surfactant complex is used to formulate a composition used to practice this invention, wherein a surfactant is associated a composition used to practice this invention via a noncovalent bond e.g. as described, e.g., in U.S. Pat. Pub. No. 20040151766.

In one embodiment, a covalent conjugate between a poly(alkylene oxide) and a glycosylated or non-glycosylated composition used to practice this invention is used, where a poly(alkylene oxide) can be conjugated to the composition via a glycosyl linking group, and a glycosyl linking group can be interposed between a composition used to practice this invention and a poly(alkylene oxide). A covalent conjugate can be formed by contacting a composition used to practice this invention with a glycosyltransferase and a modified sugar donor; the glycosyltransferase transfers the modified sugar moiety to the composition to form a covalent conjugate; the modified sugar moiety can be a poly(alkylene oxide). See e.g., U.S. Pat. No. 7,416,858.

In one embodiment, a composition used to practice this invention can be applied to cells as polymeric hydrogels or water-soluble copolymers, e.g., as described in U.S. Pat. No. 7,413,739; for example, a composition can be polymerized through a reaction between a strong nucleophile and a conjugated unsaturated bond or a conjugated unsaturated group, by nucleophilic addition, wherein each precursor component comprises at least two strong nucleophiles or at least two conjugated unsaturated bonds or conjugated unsaturated groups.

In one embodiment, a composition used to practice this invention, e.g., a neuronal-specific miR-124, can be applied to cells using vehicles with cell membrane-permeant peptide conjugates, e.g., as described in U.S. Pat. Nos. 7,306,783; 6,589,503. In one aspect, the composition itself is conjugated to a cell membrane-permeant peptide. In one embodiment, a composition and/or the delivery vehicle are conjugated to a transport-mediating peptide, e.g., as described in U.S. Pat. No. 5,846,743, describing transport-mediating peptides that are highly basic and bind to poly-phosphoinositides.

In one embodiment, electro-permeabilization is used as a primary or adjunctive means to deliver a composition of the invention to a cell, e.g., using any electroporation system as described e.g. in U.S. Pat. Nos. 7,109,034; 6,261,815; 5,874,268.

### Products of Manufacture, Implants and Artificial Organs

The invention also provides products of manufacture comprising cells of the invention, and use of cells made by methods of this invention, including for example implants and artificial organs, bioreactor systems, cell culture systems, plates, dishes, tubes, bottles and flasks comprising cells of this invention, e.g., human cells generated by practicing a method of this invention. Any implant, artificial organ, bioreactor systems, cell culture system, cell culture plate, dish (e.g., petri dish), cell culture tube and/or cell culture flask (e.g., a roller bottle) can be used to practice this invention.

In alternative embodiments the invention provides a bioreactor, implant, stent, artificial organ or similar device comprising a cell of the invention, or cells made by a method of this invention; for example, including implants as described in U.S. Pat. Nos. 7,388,042; 7,381,418; 7,379,765; 7,361,332; 7,351,423; 6,886,568; 5,270,192; and U.S. Pat. App. Pub. Nos. 20040127987; 20080119909 (describing auricular implants); 20080118549 (describing ocular implants); 20080020015 (describing a bioactive wound dressing); 20070254005 (describing heart valve bio-prostheses, vascular grafts, meniscus implants); 20070059335; 20060128015 (describing liver implants).

### Implanting Cells In Vivo

In alternative embodiments, the methods of the invention also comprise implanting or engrafting the trans-differentiated re-programmed cells (of the invention, or made by a method of this invention), or re-programmed differentiated cells (of the invention, or made by a method of this invention) in a vessel, tissue or organ; and in one aspect, comprise implanting or engrafting the re-programmed differentiated cell in a vessel, tissue or organ ex vivo or in vivo, or implanting or engrafting the re-programmed differentiated cell in an individual in need thereof.

Cells can be removed from an individual, treated using the compositions and/or methods of this invention, and reinserted (e.g., injected or engrafted) into a tissue, organ or into the individual, using any known technique or protocol. For example, trans-differentiated re-programmed cells, or re-programmed differentiated cells, can be re-implanted (e.g., injected or engrafted) using microspheres e.g., as described in U.S. Pat. No. 7,442,389; e.g., in one aspect, the cell carrier comprises a bulking agent comprising a plurality of round and smooth polymethylmethacrylate microparticles preloaded within a mixing and delivery system and an autologous carrier comprising these cells. In another embodiment, the cells are readministered to a tissue, an organ and/or an individual in need thereof in a biocompatible crosslinked matrix, as described e.g., in U.S. Pat. App. Pub. No. 20050027070.

In another embodiment, the cells of the invention (e.g., cells made by practicing the methods of this invention) are readministered (e.g., injected or engrafted) to a tissue, an organ and/or an individual in need thereof within, or protected by, a biocompatible, nonimmunogenic coating, e.g., as on the surface of a synthetic implant, e.g., as described in U.S. Pat. No. 6,969,400, describing e.g., a protocol where a composition can be conjugated to a polyethylene glycol that has been modified to contain multiple nucleophilic groups, such as primary amino or thiol group.

In one embodiment, the cells of the invention (e.g., cells made by practicing the methods of this invention) are readministered (e.g., injected or engrafted) to a tissue, an organ and/or an individual in need thereof using grafting methods as described e.g. by U.S. Pat. Nos. 7,442,390; 5,733,542.

The invention will be further described with reference to the following examples; however, it is to be understood that the invention is not limited to such examples.

### EXAMPLES

### Example 1

This invention for the first time demonstrates that inactivation of a single RNA polypyrimidine tract binding protein (PTB) is sufficient to induce the expression of a specific set of transcription factors, which act together to trigger trans-differentiation of diverse cell types into functional neurons. The inventors found that a single RNA binding protein PTB, which is naturally down regulated during brain development, is involved in regulating RNA metabolism at both the splicing and microRNA levels. The function of PTB in regulating microRNA targeting in the human genome was first demonstrated in this study. These functions cause a series of molecular switches, a most important one being the inactivation of the RE1-Silencing Transcription factor (REST; also known as Neuron-Restrictive Silencer Factor, or NRSF) complex. This leads to the induction of a series of neuronal specific genes in non-neuronal cells. In the presence of other neural trophic factors, the morphologically transformed cells become functional neurons.

Here we report that repression of a single RNA binding protein PTB, which occurs during normal brain development, is sufficient to induce trans-differentiation of fibroblasts into functional neurons. In this RNA program, neuronal-specific miR-124 targets PTB for degradation, which in turn triggers gene expression reprogramming, leading to induced expression of all critical transcription factors known to be sufficient to cause trans-differentiation of fibroblasts to neurons. Besides its established role in regulated splicing, we show that PTB has a previously undocumented function in regulating microRNA targeting. A key event in this pathway is PTB-mediated blockage of microRNA action on multiple components of the REST complex, thereby de-repressing many neuronal genes, including miR-124, in non-neuronal cells. This creates and accelerates a potent feed-forward loop to elicit cellular reprogramming to the neuronal lineage.

In PTB-depleted cells, we unexpectedly observed conversion of diverse cell types into neuronal-like cells. In addition to induced alternative splicing events, we found an extensive involvement of PTB in the regulation of microRNA targeting either through direct competition or induced switch of local RNA secondary structure. A key event is the activation of the miR-124/REST loop in which PTB not only serves as a target, but also acts as a potent regulator. Consequently, regulated PTB expression induces massive reprogramming at both the splicing and microRNA levels to drive the cell fate decision towards the neuronal lineage.

Results

PTB Down-Regulation Switches Multiple Cell Types to Neuronal-Like Cells

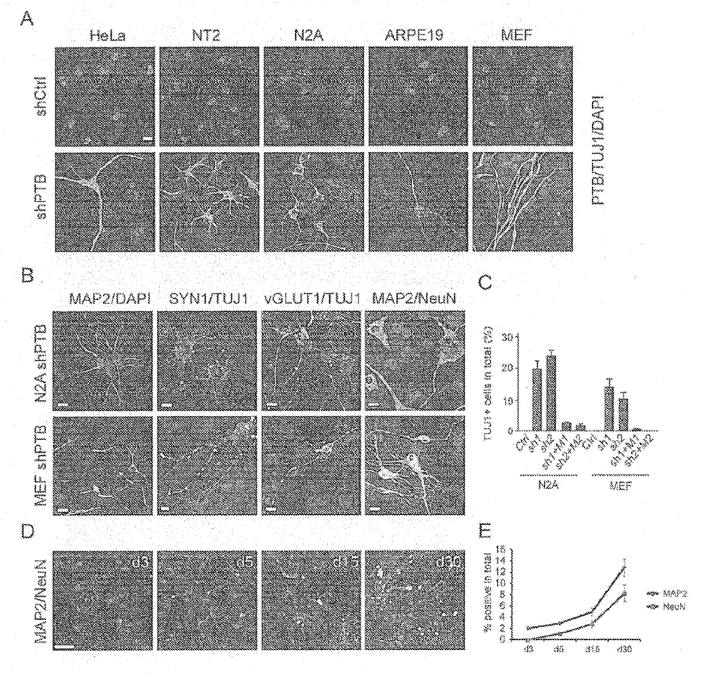

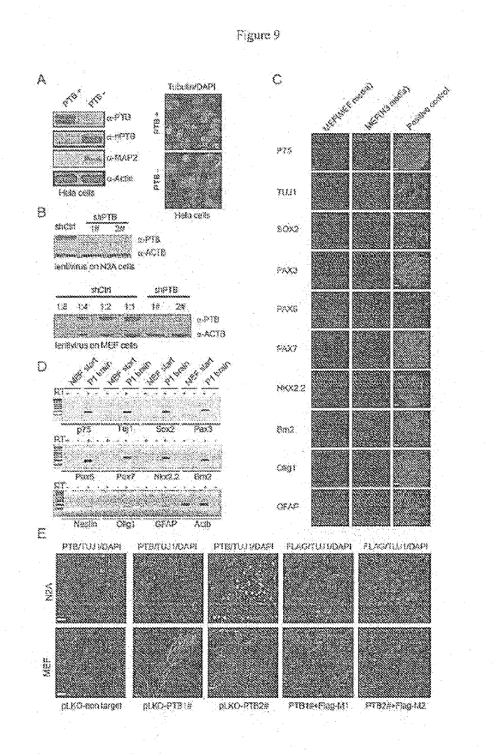

We attempted to use specific shRNAs to stably knock down PTB in order to systematically analyze PTB-regulated splicing. As expected, shPTB induced nPTB expression in HeLa cells (FIG. S1A, or FIG. 9(A)). We noted a slow growth phenotype of shPTB-treated cells, which was also seen by others (He et al., 2007). Strikingly, many PTB-depleted HeLa cells exhibited neurite outgrowth and further analysis revealed the expression of several neuron markers, including class III β-tubulin (known as Tuj1) and MAP2 (FIG. 1A and FIG. S1A), suggesting that PTB knockdown converted highly transformed HeLa cells to neuronal-like cells.

We extended this analysis to multiple cell types of diverse origin, including human embryonic carcinoma stem cells (NT2), mouse neural progenitor cells (N2A), human retinal epithelial cells (ARPE19), and primary mouse embryo fibroblasts (MEFs). Upon PTB knockdown (FIG. S1B, or FIG. 9(B)), all of these cells exhibited a neuronal-like morphology and showed strong Tuj1 staining (FIG. 1A). Neuronal committed N2A and NT2 cells were potently induced to show typical neuronal morphology in approximately 5 days after PTB knockdown and develop more complex morphology after the cells were switched to N3 media containing a set of neural growth factors for 3 to 5 days. ARPE19 and MEFs took 2 weeks to develop neuronal morphology in N3 media. Control shRNA treatment had no effect under these conditions.

We further characterized two of these cell lines (N2A and MEFs) by examining additional neural markers, including Synapsin 1 (SYN1), vGLUT1 and NeuN (FIG. 1B). SYN1 and vGLUT1 showed a typical punctate staining pattern on Tuj1-positive cells, but not on undifferentiated cells in the same field (FIG. 1B). We also detected strong staining of GABA channel receptors on these derived neurons (data not shown). As previously described (Vierbuchen et al., 2010), immunostaining and RT-PCR analyses ruled out potential contamination of our starting MEFs with neural crest cells (FIG. S1C and S1D, or FIG. 9(C) and FIG. 9(B), respectively).

Both N2A cells and MEFs were efficiently converted by two distinct shRNAs against PTB to neuronal-like cells (FIG. S1E, or FIG. 9(E)). Importantly, the effect of specific shPTB molecules could each be rescued with the shRNA-resistant PTB expression unit that carries synonymous mutations (M1 or M2) in their targeting sites, thus ruling out potential off-target effects (FIG. 1C). Time-course analysis demonstrated that PTB knockdown progressively converted MEFs to neuronal-like cells with complex morphology (FIGS. 1D and 1E). These data strongly suggest that PTB down-regulation potently induced these cells to differentiate (in the case of N2A cells) or trans-differentiate (in the case of MEFs) into neurons.

MEF-Derived Neurons are Functional with Synaptic Activities

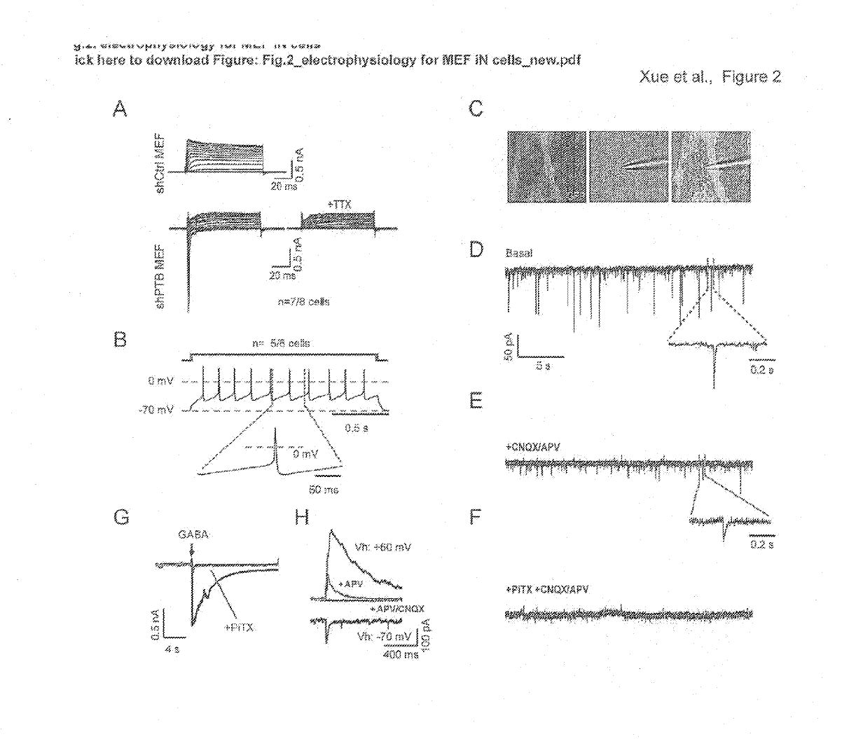

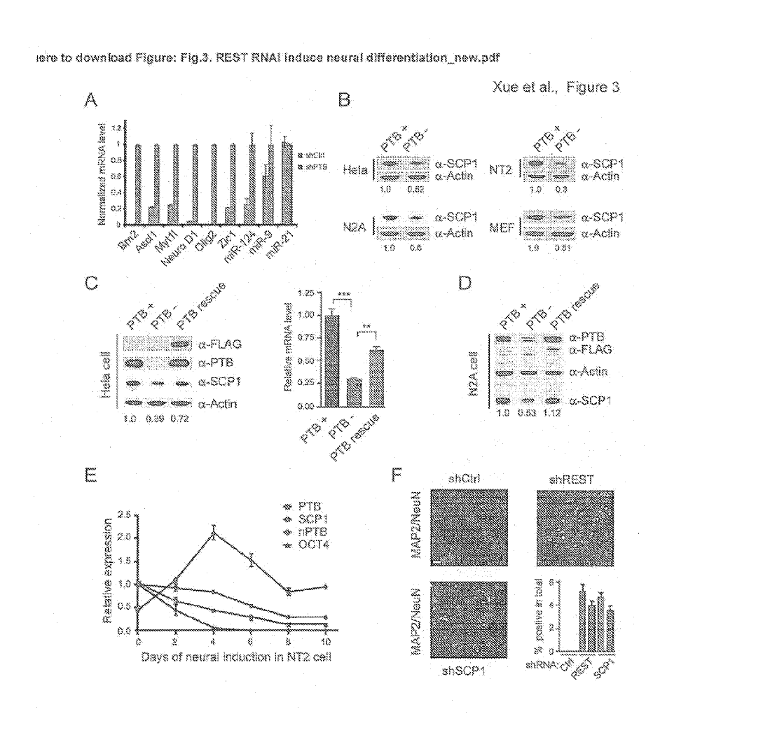

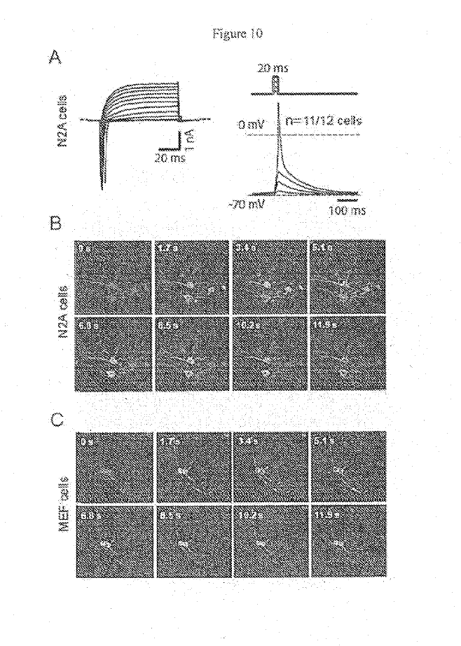

To determine the functionality of differentiated cells, we patch-clamped both shPTB-induced neurons from N2A cells and MEFs. We observed that 11 out of 12 N2A cell-derived neurons exhibited fast inward Na⁺ currents and action potential upon membrane depolarization (FIG. S2A, or FIG. 10(A)) and that 7 out of 8 shPTB-induced MEFs showed a similar response, which could be blocked by the sodium channel inhibitor TTX (FIG. 2A). Both of these induced cell types showed depolarization-induced Ca⁺⁺ influx (FIG. S2B, or FIG. 10(B) and S2C, or FIG. 10(C)). We next determined whether MEF-derived neurons are fully functional in the presence of primary astrocytes, which is known to be essential for trans-differentiated MEFs to become synaptically competent (Vierbuchen et al., 2010). After co-culture for a week with freshly isolated astrocytes free of contaminating neurons from the brain of a GFP-transgenic rat, we detected repetitive action potentials of varying frequencies driven by current pulse in 5 out of 6 MEFs-derived neurons (FIG. 2B). Importantly, we recorded synaptic activities on 6 out of 7 such neurons examined (FIGS. 2C and 2D).

The detected postsynaptic currents likely reflect both glutamatergic and GABAergic responses, because CNQX+APV (antagonists of glutamatergic channel receptors) and Picrotoxin (PiTX, antagonist of GABA_{A} channel receptors) could sequentially block the expected signals (FIGS. 2E and 2F). We further recorded GABA-induced, PiTX-sensitive currents upon focal application of GABA (FIG. 2G). In the presence of PiTX, we detected AMPA receptors-mediated excitatory postsynaptic currents (EPSC) with fast kinetics when holding the neuron at −70 mV with an external solution containing 2 mM Mg⁺⁺ (FIG. 2H), which is known to inhibit NMDA EPSC with slow kinetics (Nowak et al., 1984). By holding the neuron at +60 mV to relieve the inhibitory effect of Mg⁺⁺, we detected both NMDA and AMPA EPSCs, which could be progressively blocked by the NMDA channel inhibitor APV and the AMPA channel antagonist CNQX (FIG. 2H). These data demonstrated that shPTB had trans-differentiated MEFs into functional neurons.

PTB Regulates the Expression of Many Neuronal Genes in Non-Neuronal Cells

Because of the induced neuronal morphology and the availability of the genome-wide PTB-RNA interaction map on HeLa cells (Xue et al., 2009), we initially took this cell type as a surrogate model to understand shPTB-induced cellular reprogramming We identified by RNA-seq a large number of up- or down-regulated genes induced by shPTB (FIG. S3A) and we further confirmed a panel of these events by RT-qPCR (Figure S3B). Gene Ontology (GO) analysis showed that many such altered genes were linked to neuronal functions (FIG. S3C). These observations indicate that PTB is extensively involved in the regulation of neuronal genes in non-neuronal cells.

We noted the induction of Brn2 and Myt1l, which correspond to 2 out of 5 key transcription factors previously shown to be sufficient to induce trans-differentiation of fibroblasts into neurons (Vierbuchen et al., 2010). Because HeLa cells have a severely re-arranged genome, we performed a focused analysis on MEFs by RT-qPCR (FIG. 3A), detecting the induction of all five critical transcription factors (Ascl1, Brn2, Myt1l, Zic1, and Olig2) as well as NeuroD1 known to enhance neurogenesis in human fibroblasts (Pang et al., 2011). We also observed the induction of miR-124 and miR-9 (FIG. 3A), which have been shown to synergize with neuronal-specific transcription factors in promoting neurogenesis (Yoo et al., 2011). These data explain the compatible functionality of shPTB-induced neurons to that converted by a set of lineage-specific transcription factors.

REST Activity Contributes a Key Part to the shPTB-Induced Neuronal Program

The REST complex is known to repress a large set of neuronal genes in non-neuronal cells (Johnson et al., 2007). Interestingly, we noted that all induced transcription factors examined in FIG. 3A contain significant REST ChIP-seq signals from the ENCODE data on C2C12 cells. We confirmed strong REST binding by ChIP-qPCR on most of these genes in MEFs, and as expected, REST knockdown induced the expression of these genes (FIG. S3D and S3E). These data suggest that the function of the REST complex might be compromised in shPTB-treated MEFs.

To determine how the RSET complex was compromised, we examined the response of REST and REST co-factors to shPTB in HeLa cells. While REST expression was little affected from our RNA-seq analysis, we found that SCP1, a Pol II SerS phosphatase associated with the REST complex (Yeo et al., 2005), was significantly down regulated by shPTB in multiple cell types with induced neuronal morphology (FIG. 3B). The effect of shPTB on SCP1 expression could be rescued on two of these cell types we examined (FIGS. 3C and 3D). During the course of retinoic acid-induced neural differentiation on NT2 cells, we observed that SCP1 expression was gradually reduced, which closely tracked PTB down-regulation and nPTB induction (FIG. 3E). These findings suggest that PTB-regulated expression of the REST co-factor SCP1 may play a key role in neuronal differentiation under physiological conditions.

Recent studies suggest that REST is required for maintaining the population of neural stem cells (Gao et al., 2011) and genetic inactivation of REST does not efficiently turn fibroblasts into neurons, despite the induction of some neuronal genes (Aoki et al., 2012). However, a dominant negative SCP1 was able to efficiently drive neuronal differentiation on P19 cells (Yeo et al., 2005). We thus wished to directly test the contribution of SCP1 to shPTB-induced neurogenesis under our experimental conditions and we similarly tested REST for comparison. We found that both shSCP1 and shREST, but not control shRNA, were able to trigger neuronal differentiation on MEFs (FIG. 3F). The neural induction efficiency by shSCP1 and shREST was similar, but lower than that induced by shPTB (compared FIG. 1C and FIG. 3F), indicating that other PTB-regulated events may additionally contribute to the induction of neurogenesis. The reason for efficient induction of neurogenesis with shPTB or shRNA against REST or a REST co-factor gene may be due to gradual switch of these cell lineage-specific regulators, which may mimic relevant developmental processes (see Discussion).

PTB-Regulated Splicing Likely Facilitates the Development of the Neural Program

PTB is best known for its role in regulated splicing (Makeyev et al., 2007), which is consistent with our RNA-seq data from HeLa cells (FIG. S3F). However, it has been unclear which altered splicing event(s) contributes to the development of the neuronal lineage in PTB-depleted cells. In light of the recent finding that the REST gene itself undergoes alternative splicing to produce a truncated, non-functional isoform (REST4) (Raj et al., 2011), we asked whether this splicing event might be subjected to PTB regulation. We detected some induction of the REST4 isoform in N2A cells, but not in other cell types we examined (FIG. S3G).

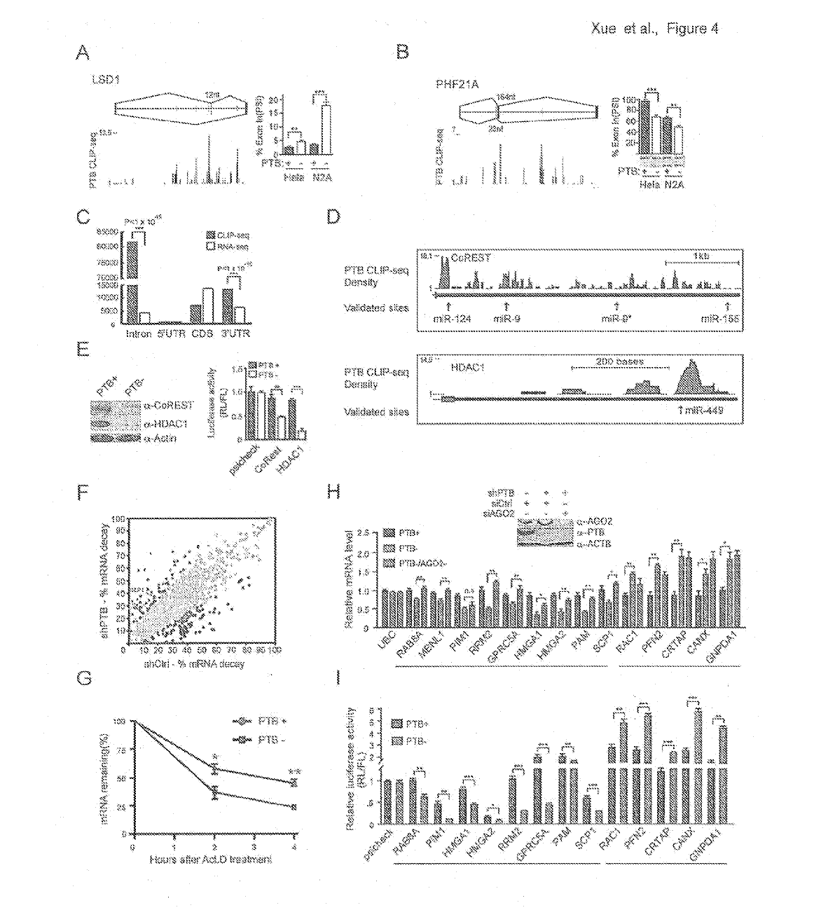

During the course of this investigation, we detected induced alternative splicing of two key genes, LSD1 (a histone lysine demethylase, a component of the REST complex) and PHF21A (a component of the histone deacetylase HDAC1 complex) upon PTB knockdown in HeLa and N2A cells (FIGS. 4A and 4B). This is consistent with multiple PTB binding events around the regulated exon in both cases from our published CLIP-seq data (Xue et al., 2009). Importantly, induced skipping of the alternative exon in LSD1 has recently been shown to affect neurite morphogenesis/maturation (Zibetti et al., 2010). Although it remains to be determined whether induced PHF21A splicing has any functional consequence, these findings suggest that some PTB-regulated splicing events may directly contribute to the neuronal phenotype observed in PTB down-regulated cells.

PTB is Involved in the RNA Stability Control of Key Neuronal Genes

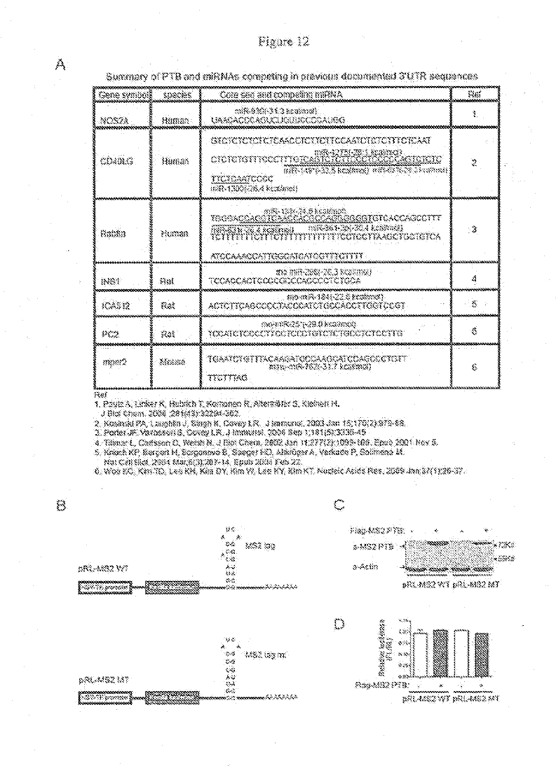

Because many PTB-affected genes could not be explained by induced splicing, we searched for other potential mechanisms. PTB has been reported to regulate RNA stability in multiple cases through C/U-rich sequences in the 3′UTR, but the mechanism has remained elusive (Knoch et al., 2004; Kosinski et al., 2003; Pautz et al., 2006; Porter et al., 2008; Tillmar and Welsh, 2002; Woo et al., 2009). By examining the PTB-RNA map (Xue et al., 2009), we noted extensive PTB binding events in the 3′UTR of all of those reported genes (FIG. S4A, or FIG. 12(A)). Globally, PTB binding on both intronic regions and 3′UTRs are more prevalent than 5′UTRs and exons compared to the RNA-seq signals in these regions (FIG. 4C). However, PTB binding alone does not seem to be sufficient to regulate RNA stability, as we showed by using an MS2-based tethering assay (FIG. S4B-4D, or FIG. 12(B), 12(C), 12(D)). We noted that all of those previously mapped PTB binding sites localize closely with predicted microRNA targeting sites (FIG. S4A, or FIG. 12(A)), raising an intriguing possibility that PTB may regulate RNA stability via functional interplay with microRNA.

Multiple PTB binding peaks are evident in the 3′UTR of CoREST and HDAC1 (FIG. 4D), both of which have been implicated in neurogenesis as key components of the REST complex (Dovey et al., 2010; Hsieh et al., 2004). These mapped PTB binding sites are coincident with three previously validated targeting sites by miR-124, miR-9 and miR-449 (Baudet et al., 2012; Packer et al., 2008; Selbach et al., 2008). Indeed, PTB knockdown in HeLa cells dramatically reduced the expression of both CoREST and HDAC1 at the protein level and diminished the luciferase activity of the reporters containing the 3′UTR of these genes (FIG. 4E). These data strongly suggest that PTB down-regulation caused dismantling of multiple components of the REST complex, which likely contribute in a collective fashion to the induction of neuronal-specific genes in non-neural cells.

PTB Regulates RNA Stability in Conjunction with microRNA

From this point, we used HeLa cells to understand the mechanism underlying PTB-regulated gene expression mainly because of the experimental manipulability of the cell type, although it is important to emphasize that caution must be taken when extrapolating deduced molecular mechanism from one cell type to another. To determine how extensively PTB is involved in RNA stability control, we performed RNA-seq on mock-depleted and PTB-depleted cells before (T₀) or after blocking transcription with Actinomycin D for 4 hours (T₄). This allowed us to calculate mRNA decay [(T₀−T₄)/T₀×100%] and determine how such decay might be influenced by PTB for each expressed gene in the human genome. We identified a total of 142 genes that showed significantly increased (red dots in FIG. 4F) or decreased (blue dots in FIG. 4F) decay (p<0.05) in response to PTB knockdown. Interestingly, SCP1 is among these genes, which was further confirmed by RT-qPCR (FIG. 4G).

We next selected a panel of PTB-bound genes to determine whether these PTB-regulated events were dependent on the microRNA machinery (FIG. 4H). We found that many PTB down-regulated genes (blue underlined in FIG. 4H) lost the response to PTB knockdown when Ago2 was inactivated. We found no or little effect on several PTB up-regulated genes after Ago2 RNAi (red underlined in FIG. 4H), consistent with the possibility that microRNA no longer acted on these genes in PTB-depleted cells. To determine whether the 3′UTR of PTB-regulated genes might mediate the response to PTB knockdown, we constructed a series of luciferase reporters containing the 3′UTR of these genes, finding that the reporters re-captured PTB-dependent suppression or enhancement (FIG. 4I). These data illustrate that PTB is involved in the regulation of RNA stability and/or translational control in conjunction with the action of microRNA on the 3′UTR of many genes.

The 3′UTR of SCP1 Contains Multiple microRNA Targeting Sites

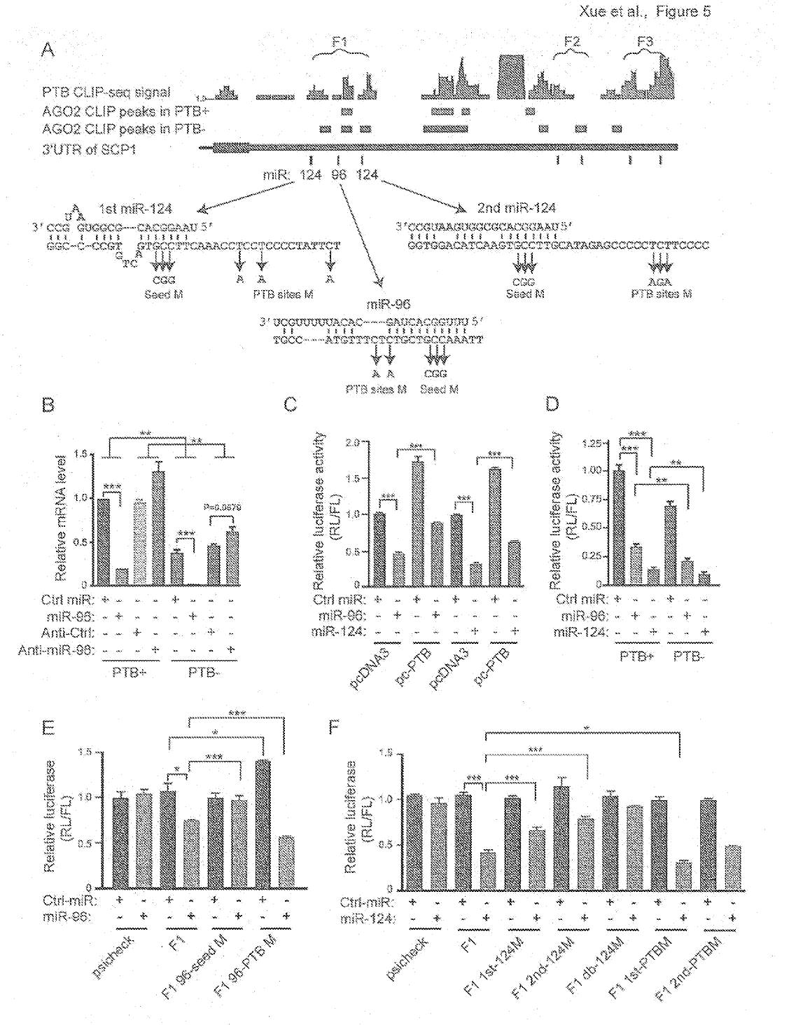

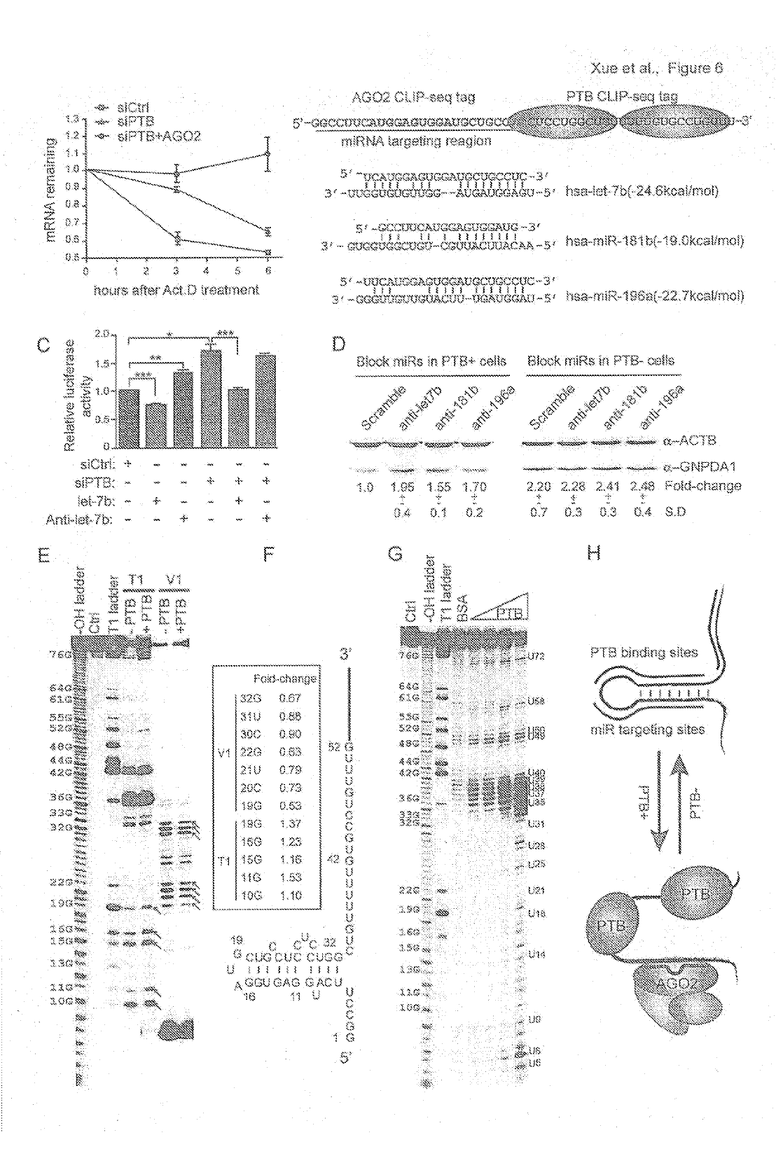

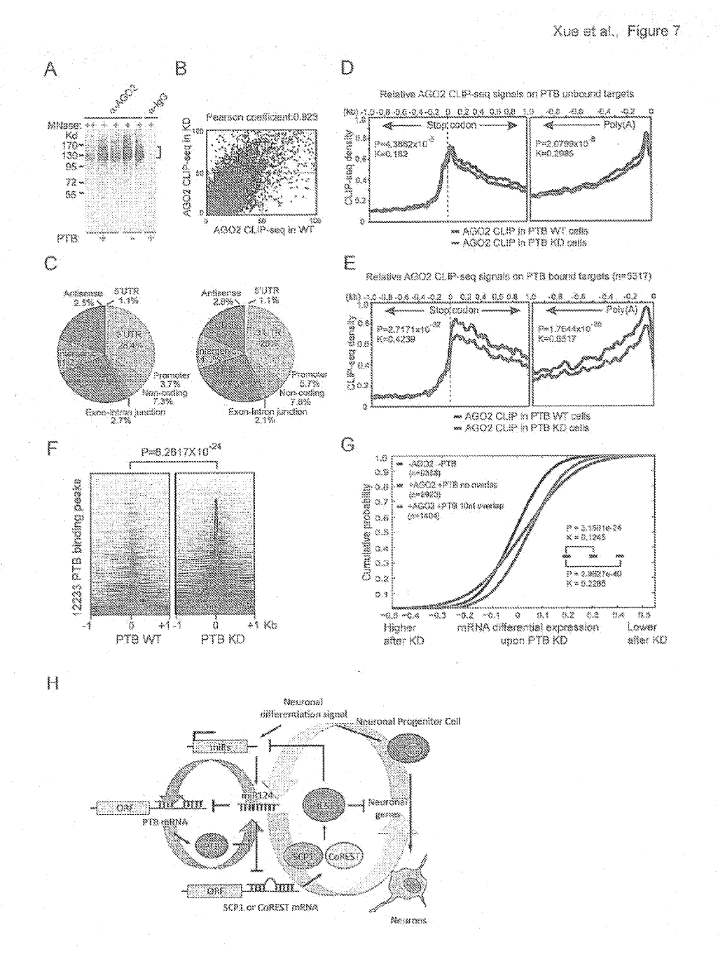

We used SCP1 as a model to investigate the functional interplay between PTB and microRNA. We compiled PTB and Ago2 CLIP-seq signals (see below in FIG. 7) in the 3′UTR of this gene before and after PTB knockdown in order to select relevant regions for functional analysis. In the F1 region, we noted three PTB binding sites (FIG. 5A): one that overlaps with the mapped Ago2 binding site before PTB knockdown (PTB+ cells) and two that become occupied by Ago2 after PTB knockdown (PTB− cells). Interestingly, one of these sites is close to the targeting site predicted for miR-96 and the other two near the predicted target sites for miR-124; each of these sites is right next to the C/U-rich PTB binding consensus. Multiple mapped PTB binding sites also overlap with the miR-124 targeting sites in the F2 and F3 regions.

Previous studies showed that forced miR-124 expression could switch the gene expression profile towards that of brain in HeLa cells (Lim et al., 2005). Relevant to the present study, miR-124 has also been shown to subject to regulation by SCP1 during neurogenesis in vivo (Visvanathan et al., 2007). Collectively, these observations suggest an important pathway for neuronal differentiation that involves the functional interplay between miR-124, PTB and SCP1/REST.

PTB Directly Competes with microRNA Targeting on the 3′UTR of SCP1

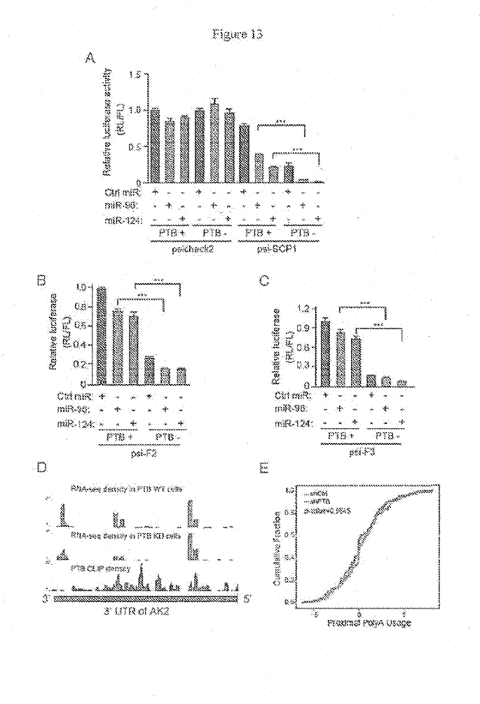

Perturbation experiments confirmed the role of PTB in the regulation of microRNA function. For example, overexpression of miR-96 suppressed SCP1 expression and PTB knockdown enhanced the effect, whereas miR-96 antagomir showed the opposite response (FIG. 5B). A non-targeting miR-339 (labeled as Ctrl miR) served as a negative control. We could recapitulate these effects with a luciferase reporter containing the entire 3′UTR of the SCP1 gene (FIG. S5A, or FIG. 13(A)). We then analyzed individual segments (F1 to F3) in the 3′UTR of SCP1, finding that overexpression of either miR-96 or miR-124 could suppress the activity of the reporter containing the F1 fragment (FIG. 5C). PTB overexpression antagonized, but PTB knockdown enhanced, the effect of both microRNAs (FIGS. 5C and 5D, or FIG. 13(C) and or FIG. 13(D)). We made a similar observation on the luciferase reporter containing the F2 (FIG. S5B, or FIG. 13(B)) or F3 (FIG. S5C, or FIG. 13(C)) fragment.

To determine the sequence requirement for both microRNA- and PTB-mediated actions, we carried out mutational analysis in the seed region of individual microRNA target sites and on the nearby PTB binding sites (FIG. 5A). We found that the mutant (GCC to CGG) in the miR-96 seed region no longer responded to the overexpression of this microRNA (FIG. 5E). The mutations (double C-to-A) in the nearby PTB binding site enhanced the effect of the microRNA, even though these mutations impaired miR-96 targeting to some degree, thus causing an increase in the reporter activity in control microRNA-treated cells (FIG. 5E). Similarly, the mutations (GCC to CGG) in each of the miR-124 targeting sites attenuated and the double mutation abolished the response to transfected miR-124 (FIG. 5F). In comparison, at least one of the mutations in nearby PTB binding sites (the triple A mutant in the first miR-124 targeting site shown in FIG. 5A) enhanced the effect of miR-124 (compare lanes 4 and 12 in FIG. 5F). Together, these data demonstrated that PTB directly competed with microRNA on multiple targeting sites in the 3′UTR of the SCP1 gene.

PTB can Also Boost microRNA Action on Specific Genes

Our RNA-seq experiments and luciferase-based assays revealed both up- and down-regulated genes in response to PTB knockdown. While many up-regulated genes likely resulted from de-repression, we detected multiple examples of up-regulated genes in PTB knockdown cells that appear to depend on their 3′UTRs (FIG. 4I). Such effect might be due to PTB-regulated switch of polyadenylation from the distal to proximal site, thereby shortening the 3′UTR in some genes that reduce microRNA targeting potentials. We tested and ruled out this possibility by measuring RNA-seq tags at the 3′ end of each expressed gene in response to PTB knockdown (FIG. S5D and S5E).

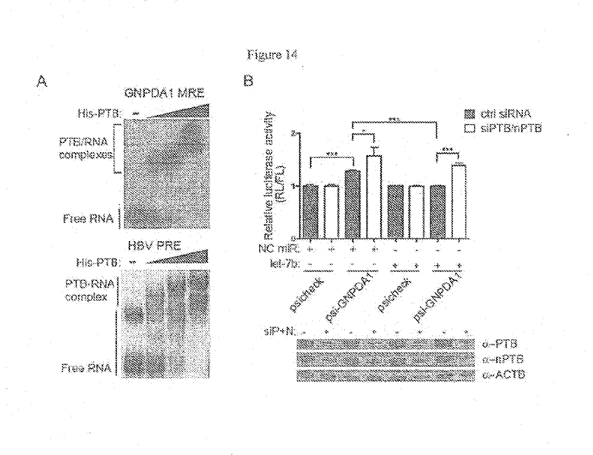

To understand how PTB knockdown could induce gene expression, we took GNPDA1 as a model, which was up regulated by PTB via its 3′UTR (FIG. 4I). We validated that PTB knockdown enhanced the stability of the endogenous GNPDA1 transcript (FIG. 6A). We noted that the CLIP-seq mapped PTB binding events are coincident with two stretches of C/U-rich sequences on the 3′UTR of the GNPDA1 gene (FIG. 6B). We confirmed high affinity PTB binding on this element by gel mobility shift (FIG. S6A, or FIG. 14(A)). Importantly, the PTB binding sites are immediately downstream of the mapped Ago2 binding sites that contain potential targeting sites for several microRNA, including Let-7b, miR-181b, and miR-196a (FIG. 6B). As expected, Let-7b overexpression suppressed the expression of the luciferase reporter containing this region, while anti-Let-7b showed the opposite effect (FIG. 6C). The reporter activity could be further enhanced by PTB knockdown in HeLa (FIG. 6C) and NT2 cells (FIG. S6B, or FIG. 14(B)). We also showed that both anti-Let-7b and anti-miR-181b enhanced GNPDA1 protein expression in a PTB-dependent manner (FIG. 6D). These data demonstrated that microRNAs act more effectively on GNPDA1 in the presence of PTB.

PTB Facilitates microRNA Action by Changing Local RNA Secondary Structure

To uncover the mechanism for PTB-dependent enhancement of microRNA action, we determined the secondary structure in the 3′UTR of GNPDA1 gene using RNase T1 to cut single-stranded RNA after the nucleotide G, and RNase V1 to cleave double-stranded RNA (FIG. 6E). This analysis suggests a stem-loop between 6U and 33G (FIG. 6F), which appears to be undertaking a dynamic switch between the single- and double-stranded states, as evidenced by T1 and V1 cleaved products in the same stem region. In the presence of PTB, we reproducibly detected enhanced single-strandness of the stem-loop, as indicated by increased T1 cleavage from 10G to 19G (red arrows in FIG. 6E) and concurrent decreased V1 cleavage from 19G to 32G (blue arrows in FIG. 6E), which were quantified on a modeled RNA structure (boxed in FIG. 6F).

We substantiated the increase of single-strandness by in-line probing, an approach widely used to detect riboswitches, which measures spontaneous RNA cleavage in solution with strong preference for U-rich residues (Regulski and Breaker, 2008). With increasing amounts of PTB, we found that the entire region gradually opened up, as indicated by enhanced cleavage on nearly all residues from 10G to 33G and the flanking U-rich PTB binding sites from 34C to 40U (FIG. 6G). Thus, PTB appears to induce the exposure of the microRNA target site through binding to multiple pyrimidine-rich regions, including that directly involved in base-paring with microRNA (FIG. 6H). In principle, such modulation of RNA secondary structure by PTB or other RNA binding proteins may enhance or shield microRNA target sites in adjacent regions, thus affecting RNA stability in both directions.

PTB Globally Regulates microRNA-mRNA Interactions in the Human Genome

To assess the global impact of PTB on both positive and negative modulation of microRNA targeting, we conducted CLIP-seq mapping of Ago2 before and after PTB knockdown in HeLa cells. As previously described (Chi et al., 2009), we detected Ago2-RNA crosslinking adducts IPed with anti-Ago2 above the position of the Ago2 protein on SDS-PAGE (FIG. 7A). We obtained ˜20 million uniquely mapped CLIP-seq tags and identified 2228 and 2041 genes that contain at least one Ago2 peak in their 3′UTRs before and after PTB knockdown, respectively. Comparison of these datasets suggests that PTB knockdown generally enhances Ago2 binding in the human genome (FIG. 7B). Ago2 binding events were significantly enriched in the 3′UTR of protein-coding genes in both mock-treated and shPTB-treated cells (FIG. 7C), especially near the stop codon and the poly(A) site (FIGS. 7D and 7E).

We next compared the relationship between PTB and Ago2 occupancies in the 3′UTR of protein-coding genes in response to PTB knockdown. The Ago2 binding profiles were similar in the protein-coding side (upstream of the stop codon) on both wild type (wt) and shPTB-treated cells, which provide important internal controls for our comparison. By separately analyzing PTB bound and unbound genes, we found that PTB depletion caused a dramatic increase in Ago2 binding in the 3′UTR of PTB bound targets, but had only a minor increase on PTB unbound targets (FIGS. 7D and 7E). These differences are highly statistically significant at the right side of the stop codon and the left side of the Poly(A) sites, as determined by two-tailed Kolmogorov-Smirnov test. This likely represents an underestimate of increased microRNA targeting events because the transcripts of many PTB bound genes were down regulated to various degrees in PTB knockdown cells. Global analysis further showed that PTB knockdown generally and significantly enhanced Ago2 binding on and around the mapped PTB binding sites (FIG. 7F). In this analysis, we noted many altered Ago2 binding events around and away from the mapped PTB binding sites, suggesting that PTB binding may have both local and long-range effects on microRNA targeting.

PTB-Regulated Ago2 Binding Functionally Correlates to Induced Gene Expression

To determine how such changes in Ago2 binding might be related to altered gene expression, we took a strategy recently used to analyze the interplay between HuR and microRNA (Mukherjee et al., 2011) to segregate expressed genes into 5 groups based on mapped PTB and Ago2 binding events in their 3′UTRs: (1) −Ago2, −PTB, (2) +Ago2, −PTB, (3) −Ago2, +PTB, (4) +Ago2, +PTB, but no overlap, and (5) +Ago2, +PTB with at least one overlapping binding event within 10 nt. This allowed us to compare gene expression changes in different groups in response to PTB knockdown by plotting genes in each group against induced transcript changes in a cumulative fashion (FIG. 7G).

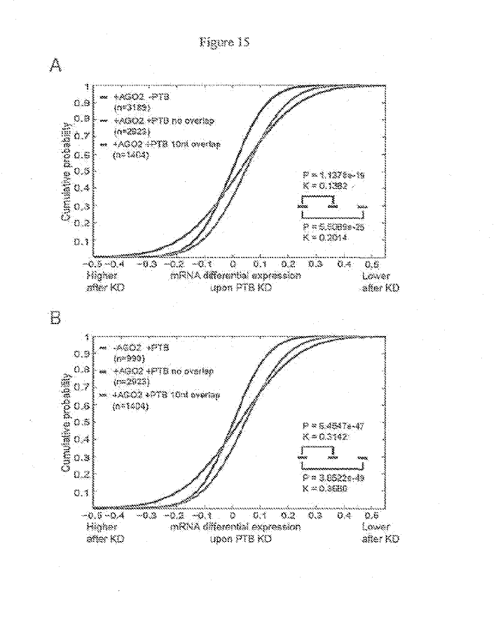

We found no significant differences between Groups 1-3, consistent with the lack of PTB and Ago2 actions on these genes. In comparison, relative to genes in Group 1 (black line), genes bound by both PTB and Ago2 but with little overlap (Group 4, green line) were linked to both repressed (right-shift at top) and enhanced gene expression (left-shift at bottom), consistent with changes in RNA secondary structure that caused increased or decreased microRNA targeting on different genes (FIG. 7G). In contrast, genes bound by both PTB and Ago2 with extensive overlap (Group 5, purple line) mainly showed repressed expression (right-shift) as a result of enhanced microRNA targeting in the absence of PTB competition (FIG. 7G). We obtained similar results in comparing genes in Group 2 (FIG. S7A, or FIG. 15(A)) and Group 3 (FIG. S7B, or FIG. 15(B)) with those in Group 4 and Group 5. These results demonstrate a large-scale involvement of PTB in regulated gene expression through its functional interplay with the microRNA machinery, which likely acts in synergy with regulated splicing to propel neurogenesis in mammalian cells.

### DISCUSSION

We now report that the reduced expression of a single RNA binding PTB, which occurs during brain development, is able to potently induce differentiation or trans-differentiation of diverse cell types into neuronal-like cells or even functional neurons.

Our data highlights the contribution of specific regulated splicing during the induction of neuronal differentiation. We discovered a PTB-regulated microRNA program responsible for dismantling of multiple components of REST. We provide further evidence that knockdown of SCP1 or the REST itself is sufficient to trigger trans-differentiation of MEFs into neuronal-like cells. The REST complex is well known for its role in suppressing many neuronal genes, including miR-124, in non-neuronal cells, while miR-124 and other neuronal specific microRNAs target various REST components, including SCP1 and CoREST. This creates a potent regulatory loop (FIG. 7H). However, this loop is inefficient, at least in the initial phase of neuronal induction, unless PTB is first down regulated by miR-124. Thus, PTB not only serves as a key target of miR-124, but also functions as a negative regulator for this and other microRNAs to act on their target genes. This represents an interesting regulatory paradigm where the large auto-regulatory loop consisting of miR-124 and components of the REST complex is controlled by another feed-forward loop that involves PTB.

Strikingly, PTB down-regulation induced the expression of all critical transcription factors previously shown to be sufficient to induce trans-differentiation of MEFs into functional neurons. Our data provide a mechanism for the induction of these transcription factors because all of these transcription factors appear to be direct REST targets. The puzzle is why genetic ablation of REST or HADC1 impaired self-renewal of neural stem cells, thus preventing unintended neurogenesis in various cellular and animal models (Dovey et al., 2010; Gao et al., 2011; Lee et al., 2002). While the cellular context undoubtedly contributes to such restriction of neurogenesis in vivo, it is possible that PTB knockdown may mimic a gradual and sequential switch of a series of events during normal developmental processes by preventing abrupt induction of gene expression that may cause cell death before differentiation. We note that the PTB-regulated RNA program takes place in cells containing induced nPTB and our preliminary results indicate that simultaneous knockdown of PTB and nPTB greatly compromised the development of neuronal morphology. This may indeed represent critical sequential events during normal brain development (Zheng et al., 2012).

Mechanistically, our study joined PTB to a growing list of RNA binding proteins, including HuR, Dnd1, CRD-BP, and PUM1, that have been implicated in modulating microRNA targeting in mammalian cells (van Kouwenhove et al., 2011). In comparison with previous studies where specific RNA binding proteins appear to either positively or negatively regulate microRNA targeting, we found that PTB can function in both ways, competing with microRNA targeting on some genes, but promoting microRNA targeting on the others. These two modes of regulation may simultaneously occur on different locations in the same genes, and thus, the net effect of positive and negative regulation may dictate the final functional outcome. These working principles may be generally applicable to many other RNA binding proteins involved in the regulation of microRNA-mRNA interactions. Our global analysis of Ago2 binding in response to PTB knockdown also suggests that PTB binding may have some long-range effects on microRNA targeting in addition to local events. This may result from potential PTB-mediated RNA looping, as proposed earlier (Oberstrass et al., 2005), the action of other induced microRNAs, or synergy with other RNA binding proteins, all of which represent interesting regulatory paradigms to be investigated in future studies.

Experimental Procedures

Cell Culture, RNAi, Immunocytochemistry and Electrophysiological Analysis

Cell culture conditions, treatments with siRNA and shRNA, and immunocytochemistry are detailed in the supplemental experimental procedures. Glial cells were isolated from GFP-transgenic rat brain (Hakamata et al., 2001) and single cell patch clamp recordings were performed using an Axopatch 200B amplifier and pClamp 10.0 software (HEKA Elektronik, Lambrecht/Pfalz, Germany), as described in the supplemental information.

RT-qPCR, Western Blotting, and Luciferase Assays

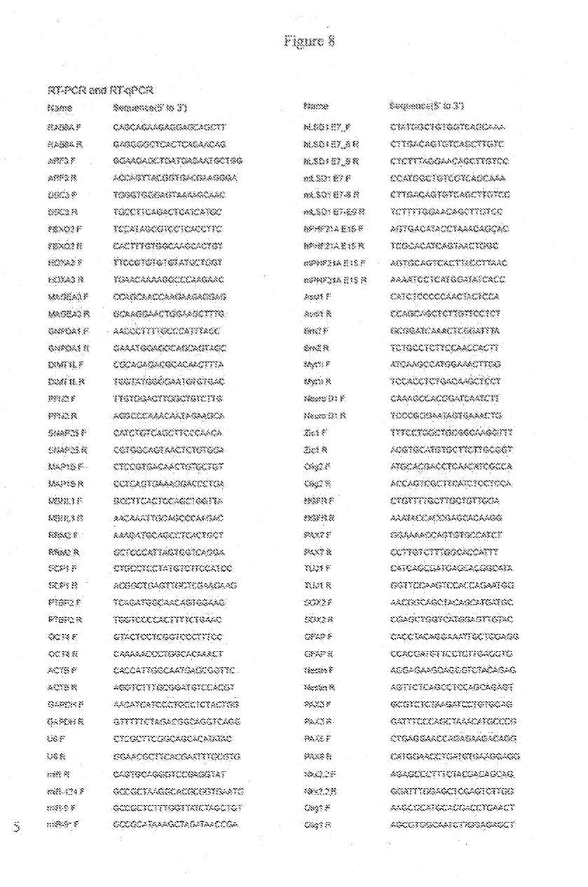

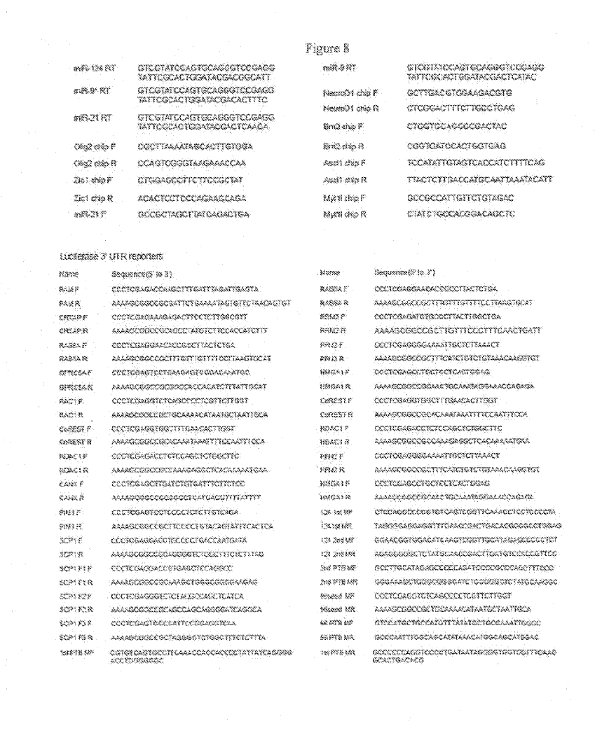

qPCR was performed with Fast Start universal SYBR green master mix using gene specific primers listed in Table 1 (FIG. 8). Luciferase activity was measured 24 hrs post-transfection. Western blotting analysis was conducted with various specific antibodies as detailed in the supplemental procedure.

RNA-Seq, CLIP-Seq, and Probing of RNA Secondary Structure

RNA-seq and CLIP-seq was performed as previously described (Xue et al., 2009). Normalized Ago2 tags are plotted relative to the stop codon at the 3′ end of genes as described (Chi et al., 2009). Two-sided Kolmogorov-Smirnov statistics (in the R package, http://cran.r-project.org/) was used to determine the significance of the shift in pair-wise comparison. RNA foot-printing by RNase T1 and V1 was according to the manual from Ambion. The in-line probing assay was as previously described (Regulski and Breaker, 2008), which is also detailed in the supplemental information.

Accession Numbers

The RNA-seq and CLIP-seq data are available at the Gene Expression Omnibus (GEO), which is a public functional genomics data repository run by NCBI, NIH; see e.g., Barrett, et al. Methods Enzymol. 2006; 411:352-69.

### REFERENCES

Amit, I., Garber, M., Chevrier, N., Leite, A. P., Donner, Y., Eisenhaure, T., Guttman, M., Grenier, J. K., Li, W., Zuk, O., et al. (2009). Unbiased reconstruction of a mammalian transcriptional network mediating pathogen responses. Science 326, 257-263.
Aoki, H., Hara, A., Era, T., Kunisada, T., and Yamada, Y. (2012). Genetic ablation of Rest leads to in vitro-specific derepression of neuronal genes during neurogenesis. Development 139, 667-677.
Auld, K. L., and Silver, P. A. (2006). Transcriptional regulation by the proteasome as a mechanism for cellular protein homeostasis. Cell Cycle 5, 1503-1505.
Ballas, N., Grunseich, C., Lu, D. D., Speh, J. C., and Mandel, G. (2005). REST and its corepressors mediate plasticity of neuronal gene chromatin throughout neurogenesis. Cell 121, 645-657.
Bartel, D. P. (2009). MicroRNAs: target recognition and regulatory functions. Cell 136, 215-233.
Baudet, M. L., Zivraj, K. H., Abreu-Goodger, C., Muldal, A., Armisen, J., Blenkiron, C., Goldstein, L. D., Miska, E. A., and Holt, C. E. (2012). miR-124 acts through CoREST to control onset of Sema3A sensitivity in navigating retinal growth cones. Nat Neurosci 15, 29-38.
Boutz, P. L., Stoilov, P., Li, Q., Lin, C. H., Chawla, G., Ostrow, K., Shiue, L., Ares, M., Jr., and Black, D. L. (2007). A post-transcriptional regulatory switch in polypyrimidine tract-binding proteins reprograms alternative splicing in developing neurons. Genes Dev 21, 1636-1652.
Caiazzo, M., Dell'anno, M. T., Dvoretskova, E., Lazarevic, D., Taverna, S., Leo, D., Sotnikova, T. D., Menegon, A., Roncaglia, P., Colciago, G., et al. (2011). Direct generation of functional dopaminergic neurons from mouse and human fibroblasts. Nature 476, 224-227.
Carracedo, A., and Pandolfi, P. P. (2008). The PTEN-PI3K pathway: of feedbacks and cross-talks. Oncogene 27, 5527-5541.
Cheng, L. C., Pastrana, E., Tavazoie, M., and Doetsch, F. (2009). miR-124 regulates adult neurogenesis in the subventricular zone stem cell niche. Nat Neurosci 12, 399-408.
Chi, S. W., Zang, J. B., Mele, A., and Darnell, R. B. (2009). Argonaute HITS-CLIP decodes microRNA-mRNA interaction maps. Nature 460, 479-486.
Conaco, C., Otto, S., Han, J. J., and Mandel, G. (2006). Reciprocal actions of REST and a microRNA promote neuronal identity. Proc Natl Acad Sci USA 103, 2422-2427.
Coutinho-Mansfield, G. C., Xue, Y., Zhang, Y., and Fu, X. D. (2007). PTB/nPTB switch: a post-transcriptional mechanism for programming neuronal differentiation. Genes Dev 21, 1573-1577.
Davis, R. L., Weintraub, H., and Lassar, A. B. (1987). Expression of a single transfected cDNA converts fibroblasts to myoblasts. Cell 51, 987-1000.
Dovey, O. M., Foster, C. T., and Cowley, S. M. (2010). Histone deacetylase 1 (HDAC1), but not HDAC2, controls embryonic stem cell differentiation. Proc Natl Acad Sci USA 107, 8242-8247.
Gao, Z., Ure, K., Ding, P., Nashaat, M., Yuan, L., Ma, J., Hammer, R. E., and Hsieh, J. (2011). The master negative regulator REST/NRSF controls adult neurogenesis by restraining the neurogenic program in quiescent stem cells. J Neurosci 31, 9772-9786.
Hakamata, Y., Tahara, K., Uchida, H., Sakuma, Y., Nakamura, M., Kume, A., Murakami, T., Takahashi, M., Takahashi, R., Hirabayashi, M., et al. (2001). Green fluorescent protein-transgenic rat: a tool for organ transplantation research. Biochem Biophys Res Commun 286, 779-785.
He, X., Pool, M., Darcy, K. M., Lim, S. B., Auersperg, N., Coon, J. S., and Beck, W. T. (2007). Knockdown of polypyrimidine tract-binding protein suppresses ovarian tumor cell growth and invasiveness in vitro. Oncogene 26, 4961-4968.
Hsieh, J., Nakashima, K., Kuwabara, T., Mejia, E., and Gage, F. H. (2004). Histone deacetylase inhibition-mediated neuronal differentiation of multipotent adult neural progenitor cells. Proc Natl Acad Sci USA 101, 16659-16664.
Johnson, D. S., Mortazavi, A., Myers, R. M., and Wold, B. (2007). Genome-wide mapping of in vivo protein-DNA interactions. Science 316, 1497-1502.
Kim, J., Efe, J. A., Zhu, S., Talantova, M., Yuan, X., Wang, S., Lipton, S. A., Zhang, K., and Ding, S. (2011). Direct reprogramming of mouse fibroblasts to neural progenitors. Proc Natl Acad Sci USA 108, 7838-7843.
Knoch, K. P., Bergert, H., Borgonovo, B., Saeger, H. D., Altkruger, A., Verkade, P., and Solimena, M. (2004). Polypyrimidine tract-binding protein promotes insulin secretory granule biogenesis. Nat Cell Biol 6, 207-214.
Kosinski, P. A., Laughlin, J., Singh, K., and Covey, L. R. (2003). A complex containing polypyrimidine tract-binding protein is involved in regulating the stability of CD40 ligand (CD154) mRNA. J Immunol 170, 979-988.
Lareau, L. F., Inada, M., Green, R. E., Wengrod, J. C., and Brenner, S. E. (2007). Unproductive splicing of SR genes associated with highly conserved and ultraconserved DNA elements. Nature 446, 926-929.
Lee, T. I., Rinaldi, N. J., Robert, F., Odom, D. T., Bar-Joseph, Z., Gerber, G. K., Hannett, N. M., Harbison, C. T., Thompson, C. M., Simon, I., et al. (2002). Transcriptional regulatory networks in Saccharomyces cerevisiae. Science 298, 799-804.
Leung, A. K., and Sharp, P. A. (2010). MicroRNA functions in stress responses. Mol Cell 40, 205-215.
Li, X., and Jin, P. (2010). Roles of small regulatory RNAs in determining neuronal identity. Nat Rev Neurosci 11, 329-338.
Lim, L. P., Lau, N. C., Garrett-Engele, P., Grimson, A., Schelter, J. M., Castle, J., Bartel, D. P., Linsley, P. S., and Johnson, J. M. (2005). Microarray analysis shows that some microRNAs downregulate large numbers of target mRNAs. Nature 433, 769-773.
Makeyev, E. V., Zhang, J., Carrasco, M. A., and Maniatis, T. (2007). The MicroRNA miR-124 promotes neuronal differentiation by triggering brain-specific alternative pre-mRNA splicing. Mol Cell 27, 435-448.
Mukherjee, N., Corcoran, D. L., Nusbaum, J. D., Reid, D. W., Georgiev, S., Hafner, M., Ascano, M., Jr., Tuschl, T., Ohler, U., and Keene, J. D. (2011). Integrative Regulatory Mapping Indicates that the RNA-Binding Protein HuR Couples Pre-mRNA Processing and mRNA Stability. Mol Cell 43, 327-339.
Nowak, L., Bregestovski, P., Ascher, P., Herbet, A., and Prochiantz, A. (1984). Magnesium gates glutamate-activated channels in mouse central neurones. Nature 307, 462-465.
Oberstrass, F. C., Auweter, S. D., Erat, M., Hargous, Y., Henning, A., Wenter, P., Reymond, L., Amir-Ahmady, B., Pitsch, S., Black, D. L., et al. (2005). Structure of PTB bound to RNA: specific binding and implications for splicing regulation. Science 309, 2054-2057.
Packer, A. N., Xing, Y., Harper, S. Q., Jones, L., and Davidson, B. L. (2008). The bifunctional microRNA miR-9/miR-9* regulates REST and CoREST and is downregulated in Huntington's disease. J Neurosci 28, 14341-14346.
Pang, Z. P., Yang, N., Vierbuchen, T., Ostermeier, A., Fuentes, D. R., Yang, T. Q., Citri, A., Sebastiano, V., Marro, S., Sudhof, T. C., et al. (2011). Induction of human neuronal cells by defined transcription factors. Nature 467, 220-223.
Pautz, A., Linker, K., Hubrich, T., Korhonen, R., Altenhofer, S., and Kleinert, H. (2006). The polypyrimidine tract-binding protein (PTB) is involved in the post-transcriptional regulation of human inducible nitric oxide synthase expression. J Biol Chem 281, 32294-32302.
Porter, J. F., Vavassori, S., and Covey, L. R. (2008). A polypyrimidine tract-binding protein-dependent pathway of mRNA stability initiates with CpG activation of primary B cells. J Immunol 181, 3336-3345.
Qiang, L., Fujita, R., Yamashita, T., Angulo, S., Rhinn, H., Rhee, D., Doege, C., Chau, L., Aubry, L., Vanti, W. B., et al. (2011). Directed conversion of Alzheimer's disease patient skin fibroblasts into functional neurons. Cell 146, 359-371.
Raj, B., O'Hanlon, D., Vessey, J. P., Pan, Q., Ray, D., Buckley, N. J., Miller, F. D., and Blencowe, B. J. (2011). Cross-Regulation between an Alternative Splicing Activator and a Transcription Repressor Controls Neurogenesis. Mol Cell 43, 843-850.
Regulski, E. E., and Breaker, R. R. (2008). In-line probing analysis of riboswitches. Methods Mol Biol 419, 53-67.
Selbach, M., Schwanhausser, B., Thierfelder, N., Fang, Z., Khanin, R., and Rajewsky, N. (2008). Widespread changes in protein synthesis induced by microRNAs. Nature 455, 58-63.
Tillmar, L., and Welsh, N. (2002). Hypoxia may increase rat insulin mRNA levels by promoting binding of the polypyrimidine tract-binding protein (PTB) to the pyrimidine-rich insulin mRNA 3′-untranslated region. Mol Med 8, 263-272.
van Kouwenhove, M., Kedde, M., and Agami, R. (2011). MicroRNA regulation by RNA-binding proteins and its implications for cancer. Nature reviews Cancer 11, 644-656.
Vierbuchen, T., Ostermeier, A., Pang, Z. P., Kokubu, Y., Sudhof, T. C., and Wernig, M. (2010). Direct conversion of fibroblasts to functional neurons by defined factors. Nature 463, 1035-1041.
Visvanathan, J., Lee, S., Lee, B., Lee, J. W., and Lee, S. K. (2007). The microRNA miR-124 antagonizes the anti-neural REST/SCP1 pathway during embryonic CNS development. Genes Dev 21, 744-749.
Watanabe, Y., Kameoka, S., Gopalakrishnan, V., Aldape, K. D., Pan, Z. Z., Lang, F. F., and Majumder, S. (2004). Conversion of myoblasts to physiologically active neuronal phenotype. Genes Dev 18, 889-900.
Woo, K. C., Kim, T. D., Lee, K. H., Kim, D. Y., Kim, W., Lee, K. Y., and Kim, K. T. (2009). Mouse period 2 mRNA circadian oscillation is modulated by PTB-mediated rhythmic mRNA degradation. Nucleic Acids Res 37, 26-37.
Xue, Y., Zhou, Y., Wu, T., Zhu, T., Ji, X., Kwon, Y. S., Zhang, C., Yeo, G., Black, D. L., Sun, H., et al. (2009). Genome-wide analysis of PTB-RNA interactions reveals a strategy used by the general splicing repressor to modulate exon inclusion or skipping. Mol Cell 36, 996-1006.
Yang, N., Ng, Y. H., Pang, Z. P., Sudhof, T. C., and Wernig, M. (2011). Induced neuronal cells: how to make and define a neuron. Cell Stem Cell 9, 517-525.
Yeo, M., Lee, S. K., Lee, B., Ruiz, E. C., Pfaff, S. L., and Gill, G. N. (2005). Small CTD phosphatases function in silencing neuronal gene expression. Science 307, 596-600.
Yoo, A. S., Sun, A. X., Li, L., Shcheglovitov, A., Portmann, T., Li, Y., Lee-Messer, C., Dolmetsch, R. E., Tsien, R. W., and Crabtree, G. R. (2011). MicroRNA-mediated conversion of human fibroblasts to neurons. Nature 476, 228-231.
Zernicka-Goetz, M., Morris, S. A., and Bruce, A. W. (2009). Making a firm decision: multifaceted regulation of cell fate in the early mouse embryo. Nat Rev Genet 10, 467-477.
Zheng, S., Gray, E. E., Chawla, G., Porse, B. T., O'Dell, T. J., and Black, D. L. (2012). PSD-95 is post-transcriptionally repressed during early neural development by PTBP1 and PTBP2. Nat Neurosci 15, 381-388, 5381.
Zibetti, C., Adamo, A., Binda, C., Forneris, F., Toffolo, E., Verpelli, C., Ginelli, E., Mattevi, A., Sala, C., and Battaglioli, E. (2010). Alternative splicing of the histone demethylase LSD1/KDM1 contributes to the modulation of neurite morphogenesis in the mammalian nervous system. J Neurosci 30, 2521-2532.

### FIGURE LEGENDS

FIG. 1. Differentiation of Diverse Cell Types into Neuronal-Like Cells in Response to PTB Knockdown

(A) Induction of neuronal morphology and the expression of the neuronal marker Tuj1 in multiple cell types in response to depletion of PTB. Scale bar: 20 p.m.

(B) Characterization of two cell types (N2A and MEF) with additional neural markers. Typical punctate staining is evident (yellow) with antibodies against Synapsin and vGLUT1. Scale bar: 20 p.m.

(C) Quantification of induced neuronal-like cells derived from N2A and MEFs. The data were based on positive Tuj1 stained cells divided by initial plating cells in response to two separate shPTBs (sh1 and sh2). The effect could be efficiently rescued with the corresponding shRNA-resistant PTB expression units that contain mutations in the corresponding target sites (M1 and M2). Data are shown as mean±SD.

(D) Time course analysis of neuronal induction on shPTB-treated MEFs. MAP2 and NeuN were stained at indicated time points. Scale bar: 60 μm

(E) Quantified temporal profile of PTB knockdown-induced neurons. Data shown as mean±SD are based on 4 equivalent areas shown in D. See also FIG. 51 (FIG. 9).

FIG. 2. Synaptic Activities on Neurons Derived from shPTB-Induced MEFs

(A) Representative traces of whole-cell currents on control shRNA-treated (top) and shPTB-treated (bottom) MEFs. Only shPTB-treated MEFs exhibited fast inward sodium currents, which could be blocked by 1 μM sodium channel inhibitor TTX.

(B) Representative trace of action potentials in response to step current injections on shPTB-induced neurons after co-culturing with rat glial cells.

(C) Image of an shPTB-induced neuron co-cultured with GFP-marked rat glial cells. Recording electrode was patched on the shPTB-induced neuron (middle and right).

(D to F) Representative traces of spontaneous postsynaptic currents on shPTB-induced neurons (D). The cell was held at −70 mV, revealing events of various amplitudes and frequencies. The insert shows a representative trace of synaptic response. Glutamategic synaptic currents were blocked with 20 μM CNQX plus 50 μM APV (E). The insert highlights the remaining GABA current. GABA currents were blocked with 50 μM PiTX (F).

(G) Induction of GABA currents by focal application of 1 mM GABA, which could be blocked by PiTX (red).

(H) Representative trace of synaptic currents recorded on shPTB-induced neurons. Vh: holding potential. AMPA-R mediated EPSC was recorded at −70 mV. Blockage of Mg⁺⁺ to NMDA-R was relieved at +60 mV, revealing both AMPA and NMDA EPSCs, which could be sequentially blocked with 50 μM APV (antagonist of NMDA-type glutamate receptors) and 20 μM CNQX (antagonist of AMPA receptors).

The number of cells that show the representative response against total cells examined is indicated in each panel. See also FIG. S2, or FIG. 10.

FIG. 3. De-Repression of Neuronal-Specific Genes in Response to PTB Knockdown

(A) RT-qPCR analysis of a panel of transcription factors and microRNAs in shPTB-treated MEFs. Data are normalized against Actin; miR-21 served as a negative control.

(B) Down-regulation of SCP1 in multiple cell types determined by Western blotting.

(C and D) Rescue of SCP1 expression in PTB knockdown cells by an shRNA-resistant PTB in HeLa (C) and N2A (D) cells.

(E) Time course analysis of neural induction by retinoic acid (RA) on NT2 cells analyzed by RT-qPCR. Oct4 was analyzed as a control. Data are shown as mean±SD.

(F) Induction of neuronal differentiation on MEFs with shRNA against SCP1 or REST. The induction efficiency was calculated based on the number of cells with positive MAP2 and NeuN staining divided by total plating cells. Data are shown as mean±SD. See also FIG. S3.

FIG. 4. PTB-Regulated Splicing and RNA Stability

(A and B) PTB-regulated alternative splicing of LSD1 and PHF21A. The CLIP-seq mapped PTB binding events (blue) are shown along with deduced PTB binding peaks (orange lines) on each gene model. PTB knockdown induced alternative splicing was determined by RT-qPCR in the case of LSD1 and by semi-quantitative RT-PCR in the case of PHF21A.

(C) Relative enrichment of PTB binding in intronic and 3′UTR regions. Significant enrichment of PTB binding events is indicated by the p-values in each case.

(D) PTB binding on two REST component genes, showing that multiple PTB binding peaks overlap with validated targeting sites by miR-124 and miR-9.

(E) Reduced CoREST and HDAC1 proteins (left) and diminished reporter activities (right) in PTB-depleted HeLa cells.

(F) Genome-wide analysis of PTB-regulated RNA stability. The calculated decay rate was compared in the presence (shCtrl-treated) or absence (shPTB-treated) of PTB. Genes with increased and decreased decay are highlighted in red and blue, respectively, based on triplicated RNA-seq data (p<0.05).

(G) Accelerated SCP1 mRNA decay detected by RT-qPCR in PTB-depleted HeLa cells.

(H) The effect of knocking down PTB (PTB-) or both PTB and Ago2 (PTB-/Ago2-) on the expression of a panel of genes that show PTB and Ago2 binding events in their 3′UTRs. A gene (UBC) without binding evidence for PTB and Ago2 severed as a negative control.

(I) Re-capture of PTB-dependent regulation with the 3′UTR of individual genes analyzed in H. Note that the MBNL1 gene was not included in this analysis because its 3′UTR is too long to clone.

Data in individual panels are shown as mean±SD. **p<0.01; ***p<0.001. See also FIG. S4, or, or FIG. 12.

FIG. 5. PTB Competition with microRNA Targeting in the 3′UTR of SCP1

(A) The mapped PTB binding events in the 3′UTR of the SCP1 gene (top). Above the gene model show the mapped Ago2 binding peaks before (red) and after (black) PTB knockdown in HeLa cells. Below the gene model indicate multiple predicted microRNA target sites for miR-124 (brown lines) and miR-96 (cyan lines). Arrow-highlighted are deduced base-paired regions between the mRNA and individual microRNAs. Also illustrated are the mutations in the 3′UTR of the SCP1 gene that correspond to the sequence on the microRNA targeting sites in the seed region (violet) or on the PTB binding site (red) in each case.

(B) The effects on the endogenous SCP1 mRNA by overexpressed miR-96 and its antagomir before and after PTB knockdown.

(C) Blockage of the effect of overexpressed miR-96 and miR-124 by PTB overexpression on the luciferase reporter containing the F1 fragment from the SCP1 3′UTR.

(D) Enhanced effect of overexpressed miR-96 and miR-124 in response to PTB knockdown on the luciferase reporter containing the F1 fragment from the SCP1 3′UTR.

(E) The requirement for the seed region in the miR-96 target site to respond to overexpressed miR-96. While the mutations in the PTB binding site impaired miR-96 targeting (compared lanes 3 and 7), the mutants enhanced the overall effect of miR-96 on the luciferase reporter (compare lanes 3/4 and lanes 7/8).

(F) Contribution of individual miR-124 target sites in the SCP1 F1 region to microRNA-mediated down-regulation of the luciferase activity. The mutations in the seed region of miR-124 targeting sites progressively reduced the response to overexpressed miR-124 (compare lanes 3 to 10). The mutations in the PTB binding site near the first miR-124 targeting sites enhanced miR-124 mediated down-regulation (compare lanes 4 and 12).

The statistical significance in comparing different groups was determined by paired t-test. Data in individual panels are shown as mean±SD. **p<0.01; ***p<0.001. See also FIG. S5.

FIG. 6. Enhanced microRNA Targeting by Modulating RNA Secondary Structure

(A) Stabilization of the GNPDA1 transcript in response to PTB and/or Ago2 knockdown in the presence of the transcription inhibitor Act.D.
(B) Potential microRNA targeting sites near the mapped PTB binding site in the 3′UTR of GNPDA1.

(C) Overexpressed Let-7b suppressed and antagomir Let-7b enhanced the expression of the luciferase reporter containing the 3′UTR of GNPDA1 (lanes 1 to 3). PTB knockdown enhanced the luciferase activity (compared between lanes 1 and 4). Overexpression of Let-7b still suppressed the luciferase activity, but anti-Let-7b no longer showed the effect in PTB knockdown cells.

(D) Antagomir Let-7b, miR-196a and miR-181b increased GNPDA1 protein in the presence, but not absence, of PTB in transfected HeLa cells. The protein levels were quantified with the SD shown in the bottom.

(E and F) Mapping the secondary structure in the 3′UTR of GNPDA1. Individual G residues were labeled on the left with red indicating several key positions in the deduced secondary structure (E), as modeled (F). Red and blue arrows respectively indicate PTB enhanced and suppressed cleavages in the deduced stem-loop region. Quantified fold-changes at key positions are indicated in the box inserted in panel F.

(G and H) Increased single-strandness of RNA in the presence of increasing amounts of PTB detected by in-line probing (G). A proposed model indicates PTB-mediated opening of the stem-loop that facilitates microRNA targeting (H).

Data in A, C, and D are shown as mean±SD. *p<0.05; **p<0.01; ***p<0.001. See also FIG. S6, or FIG. 14.

FIG. 7. Global Analysis of Ago2 Binding in Response to PTB Knockdown

(A) CLIP signals detected with anti-Ago2 before and after PTB knockdown. No signal was detected with IgG control.

(B) Comparison between the two Ago2 CLIP-seq datasets in 1 kb windows across the human genome before and after PTB depletion.

(C) Genomic distribution of Ago2 binding events before (left) and after (right) PTB knockdown, showing prevalent Ago2 binding in the 3′UTR region.

(D and E) Ago2 binding in the 3′UTR of PTB unbound (D) and bound (E) targets before (red) and after (blue) PTB knockdown. Dramatic differences were detected on PTB bound targets (n=5317) in E, which compares to much less responses on a similar number of randomly selected PTB unbound targets in D. Statistical significance was determined for the differences on both the stop codon and poly (A) sides by two-tailed Kolmogorov-Smirnov test with both the p- and k-values shown in the insert.

(F) Induction of significant Ago2 binding on and near the PTB binding sites. The p-value for the differences is indicated on the top.

(G) Functional correlation between PTB/microRNA interplay and gene expression. Genes with induced and repressed expression are plotted in a cumulative fashion. Statistical significance was determined by KS-test.

(H) Model for the PTB-regulated miR124-REST loop. See also FIG. S7, or FIG. 15.

FIG. 9, or FIG. S1, is related to main FIG. 1, showing the induction of neuronal phenotype in response to PTB knockdown in multiple cell types:

(A) Western blotting analysis showing the induction of nPTB as well as a neuronal marker MAP2 in PTB knockdown HeLa cells (left). HeLa cells depleted of PTB exhibited neurite outgrowth (right).

(B) Efficient knockdown of PTB with two different shPTBs in MEFs and N2A cells.

(C) Evidence for the lack of contaminating neurons or neural crest cells based on immunostaining for a large number of neural markers as shown. Each antibody was individually validated using appropriate positive controls, including neural progenitors isolated from E14.5 mouse brain, which were stained for P75, Pax3, Pax7, NKX2.2, Brn2 and Olig1; shPTB-induced MEFs for Tuj1; human fetal retinal progenitor for Sox2 and Pax6; and mouse muller glial cells for GFAP.

(D) Evidence for the lack of contaminating neurons or neural crest cells based on RT-PCR analysis against a large panel of neural specific genes.

(E) Induction of neuronal differentiation in both N2A and MEFs with two different shRNAs against PTB (PTB#1 and PTB#2) and rescue of the phenotype with specific shRNA-resistant, FLAG tagged PTB expression units (FLAG-M1 and FLAG-M2) that contain synonymous mutants in each shPTB targeting site. Note that the immunostaining was done on transfected cells without drug selection. In these experiments, MEFs were transfected with a much lower efficiency in comparison with N2A cells.

FIG. 10, or FIG. S2 is related to main FIG. 2, showing neural activities in shPTB-induced neurons:

(A) Representative traces of whole-cell currents in a voltage-clamp mode and depolarization-induced single action potential on induced neuronal like cells derived from N2A cells.

(B) Rapid Ca⁺⁺ influx was measured using Fluo-S-AM in response to membrane depolarization on shPTB-induced neuronal like cells from N2A cells. Cell images in time sequence (second) were shown.

(C) Rapid Ca⁺⁺ influx was measured using Fluo-S-AM in response to membrane depolarization on shPTB-induced neuronal like cells from MEFs.

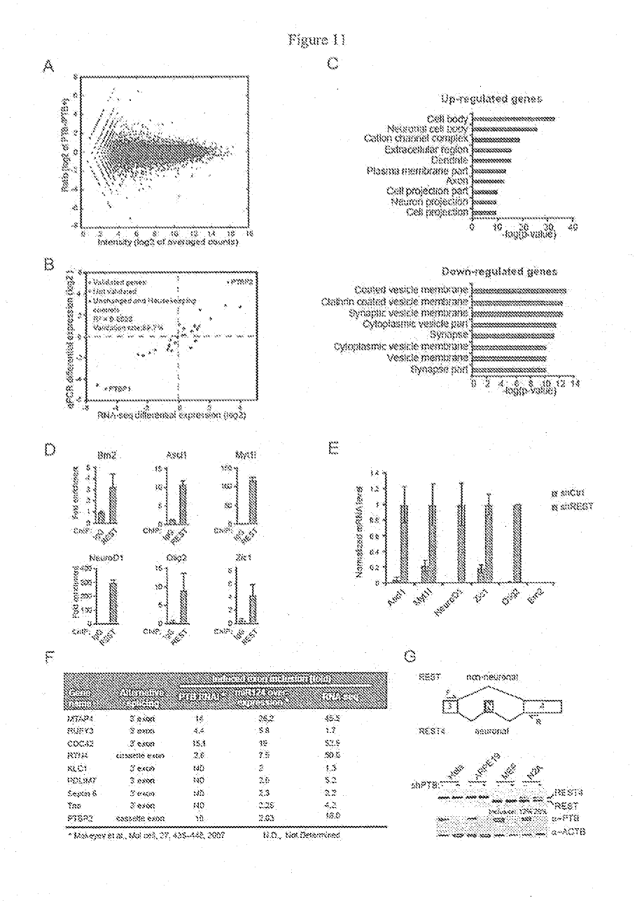

FIG. 11, or FIG. S3, is related to main FIG. 3, illustrating altered expression of many neuronal-specific genes in PTB knockdown cells:

(A) RNA-seq analysis of gene expression in response to PTB knockdown in HeLa cells. Significantly up- and down-regulated genes were labeled red and blue, respectively, with green dots representing those that have neuronal-related functions documented in literature.

(B) RT-qPCR validation of a panel of genes that were altered to different degrees (blue) as well several housekeeping genes (purple) in response to PTB knockdown in HeLa cells. The data were plotted against the RNA-seq results. Red indicates three cases where the qPCR results were not consistent with the RNA-seq results.

(C) Gene Ontology (GO) analysis of PTB-regulated genes. Top enriched GO terms (−log₂(p)>10) are highlighted for both up-regulated (red) and down-regulated (blue) genes that are related to neuronal functions.

(D) Confirmation of REST binding on a panel of shPTB-induced genes by ChIP-qPCR on MEFs. IgG was test as a control.

(E) Induction of multiple neuronal specific genes in MEFs treated with REST RNAi. Note that Brn2 was not induced after 72 hours shREST treatment, indicating that PTB may regulate Brn2 through another mechanism(s).

(F) Comparison between PTB-regulated splicing events previously reported (Makeyev et al., 2007) and their splicing changes in PTB knockdown cells determined by RNA-seq in the present study.

(G) REST splicing. Inclusion of the neuronal exon (N) will result in the production of the REST4 isoform, which encodes a truncated, non-functional REST protein. PTB knockdown induced N inclusion to some extent on N2A cells, but not in other cell types examined

FIG. 12, or FIG. S4 is related to main FIG. 4, showing the role of PTB in the regulation of pre-mRNA splicing and microRNA targeting:

(A) Previously reported cases of PTB-regulated RNA stability that contain predicted microRNA targeting sites on the mapped PTB binding sites. References for individual reported cases are listed below.

(B) Illustration of the MS2 tethering approach. We first introduced a phage RNA binding motif (MS2) to the 3′UTR of a luciferase reporter. A mutant MS2 motif containing a point mutation known to disrupt binding by the MS2 RNA binding domain served as a negative control. This permits tethering of PTB to target RNA by expressing an MS2-PTB fusion protein in co-transfected HeLa cells.

(C) Similar levels of the PTB-MS2 fusion protein expressed in HeLa cells co-transfected with wild type and mutant reporters.

(D) Lack of influence of overexpressed PTB-MS2 fusion protein on the luciferase activity, indicating that PTB binding alone may not be sufficient to alter RNA stability.

FIG. 13, or FIG. S5 is related to main FIG. 5, showing PTB regulation of microRNA targeting in the F2 and F3 fragments from the 3′UTR of SCP1 and the effect of PTB in causing 3′ end switch:

(A-C) Luciferase reporter assays on the entire SCP1 3′UTR (A), the F2 fragment from the SCP1 3′UTR (B) and the F3 fragment from the SCP1 3′UTR (C). Overexpression of miR-96 or miR-124 repressed the reporter activity and PTB knockdown further enhanced the effects. Data are shown as mean±SD. ***p<0.001.

(D) PTB-induced switch in alternative polyadenylation. We tested the possibility that regulated polyadenylation might explain some microRNA-induced isoform switches. By taking advantage of the RNA-seq data we generated that measure digital tags at the 3′ end of each expressed gene in the genome (Fox-Walsh et al., 2011), we measured alternative polyadenylation events induced by PTB knockdown. Among 6166 genes that showed detectable cleavage sites, 324 genes exhibit more than one polyadenylation event (requiring more than 10 counts on all cleavage sites). Out of these 324 genes, 14 genes showed induced switch in response to PTB knockdown, one of which is illustrated in this panel. We note that all PTB knockdown-induced events are switches from the distal to proximal cleavage site. These likely resulted from enhanced microRNA actions that degraded the longer isoform in each case.

(E) Statistical analysis based on two-sided Kolmogorov-Smirnov test indicates that PTB knockdown caused little global changes in alternative polyadenylation.

FIG. 14, or FIG. S6 is related to main FIG. 6, demonstrating high affinity PTB binding to the 3′UTR region of the GNPDA1 gene:

(A) Gel shift analysis of PTB binding on the mapped PTB binding site near the microRNA regulatory element (MRE) in the 3′UTR of the GNPDA1 gene. This binding affinity is similar to a well-characterized PTB binding site in the HBV genome (Huang et al., 2011). Final PTB concentrations in each experiment were 0, 0.35, 0.7, and 1.4 μM.

(B) The 3′UTR of the GNPDA1 gene was cloned into a luciferase reporter. The reporter activity was increased in response to double knockdown of PTB and nPTB in NT2 cells without (compare between lanes 3 and 4) or with Let-7b overexpression (compare between lanes 7 and 8). Western blotting validated the knockdown efficiency of PTB and nPTB (bottom).

FIG. 15, or FIG. S7 is related to main FIG. 7, revealing that both increased and decreased gene expression are linked to PTB and Ago2 binding events

(A) Comparison of genes in group 2 (blue line, with binding evidence for Ago2, but not PTB) with genes in group 4 (green line) that showed both Ago2 and PTB binding, but little overlap between their binding events, and with genes in group 5 (purple line) that exhibited overlapped binding events between Ago2 and PTB (at least one pair of peaks separated by <10 nt).

(B) Comparison of genes in group 3 (coffee-colored line that showed binding evidence for PTB, but not Ago2) with genes in group 4 and 5. Gene numbers in each group were indicated and p- and k-values based on two-sided Kolmogorov-Smirnov test are shown in each panel.

For Studies Illustrated in Supplemental Figures, or FIGS. 9 to 15

Cell Culture, RNAi, and Immunocytochemistry

HeLa cells were maintained in Dulbecco's Modified Eagle Medium (DMEM) containing 10% FBS (Omega Scientific) and 100 U of penicillin/streptomycin (Life Technology). NT2 cells were cultured in Minimum Essential Medium (MEMα, which contains ribonucleosides, deoxyribonucleosides and GlutaMAX™) plus 10% FBS and 100 U of penicillin/streptomycin. N2A cells were propagated in DMEM supplemented with 10% FBS, 100 U of penicillin/streptomycin. Mouse Embryonic Fibroblasts (MEFs) were isolated from E14.5 C57/BL6 mouse embryos. Head, vertebral column, and all internal organs were removed and the remaining embryonic tissues were manually dissociated followed by incubation in 0.25% Trypsin (Life Technology) for 10 min. MEFs were cultured in DMEM plus 10% FBS, non-essential amino acids, sodium pyruvate, and penicillin/streptomycin. ARPE19 cells were cultured in DMEM/F12 plus 10% FBS, 1% non-essential amino acids, and 100 U of penicillin/streptomycin.

Lentiviral shRNAs against human PTBP1 (TRCN0000231420, TRCN0000001062), mouse PTBP1 (TRCN0000109272, TRCN0000109274), Mouse REST (TRCN0000321488, TRCN0000071346), mouse CoREST (TRCN0000071368, TRCN0000071371) and mouse CTDSP1 (which encodes for SCP1) were purchased from Thermo Scientific and cloned in the pLKO.1 vector. Individual shRNAs were packaged into replication-incompetent lentiviral particles in HEK293T cells by co-transfecting individual pLKO plasmids with the packaging mix (Sigma). Viral particles were collected twice 48 hrs and 72 hrs post-transfection. Cells were infected with individual lentiviral particles for 16 hrs followed by selection with 2 μg/ml Puromycin for 48 hrs.

Selected cells were switched to different media to allow further development of complex neuronal morphology: HeLa and NT2 cells were switched to N3 media (DMEM/F12 plus 25 μg/ml insulin, 50 g/ml transferring, 30 nM sodium selenite, 20 nM progesterone, 100 nM putrescrine) or N3 media supplemented with a panel of neurotrophic factors, including BDNF, GDNF, NT3 and CNTF (Peprotech) and Ara-C (2 μM, Sigma). MEF and ARPE19 cells were first cultured in N3 media plus FGF2 (10 ng/ml) for 3 days, switched to N3 media for a week to 10 days, and then supplemented with BDNF, GDNF, NT3 and CNTF (Peprotech) for additional 6 days before immunocytochemical and electrophysiological analyses. N2A cells were maintained in DMEM supplemented with 10% FBS, 100 U of penicillin/streptomycin and 1 μg/ml Puromycin (Clontech). The media were then supplemented with BDNF, GDNF, NT3 and CNTF for 3 days prior to electrophysiological analyses. It is important to emphasize that none of the cell types cultured under above described conditions exhibited neurite outgrowth when treated with a control shRNA.

Immunocytochemistry experiments were performed on cells seeded on coverslip that had been coated with poly-D-lysin (0.05 mg/ml) and laminin (0.005 mg/ml) overnight at 37° C. Cells were washed twice with PBS, fixed in 4% Paraformaldehyde (Wako) for 15 min at room temperature, and permeabilized with 0.1% Triton X-100 in PBS for 15 min on ice. After washing three times with PBS, cells were blocked in PBS containing 3% BSA for 1 hr at room temperature.

The following primary antibodies with indicated dilution in blocking buffer were used: Rabbit anti-Tuj1 (Covance, 1:1,000), Mouse anti-Tuj1 (Covance, 1:1,000), Rabbit anti-MAP2 (Cell Signaling Technology, 1:200), Mouse anti-NeuN (Milipore, 1:200), Rabbit anti-Synapsin I (Sigma, 1:1000), Rabbit anti-Synapsin I (Milipore, 1:500), Rabbit anti-VGLUT1 (Synaptic Systems, 1:200), Rabbit anti-GABA (Sigma, 1:1000), Mouse anti-PSD95 (NeuroMab, 1:100), Rabbit anti-NGF receptor P75 (Milipore, 1:100), Rabbit anti-Brn2/POU3F2 (Cell Signaling Technology, 1:200), goat anti-Sox2 (Santa cruz, 1:200), Mouse anti-Pax3 (DSHB, 1:250), Mouse anti-Pax6 (Covance, 1:100), Mouse anti-Pax7 (DSHB, 1:250), Mouse anti-NKX2.2 (DSHB, 1:100), Mouse anti-Olig1 (Neuromab, 1:100), Mouse anti-GFAP (Neuromab, 1:100), Mouse anti-CoREST (BD biosciences, 612146), Rabbit anti-CTDSP1 (Sigma, SAB4502550). After staining with corresponding secondary antibodies in PBS plus 1% BSA, coverslips were washed six times with PBS, each for 5 min, mounted with the mounting medium containing DAPI (Vector Labs) onto glass slides, and examined under Olympus FluoView FV1000.

Glial Cell Isolation and Electrophysiological Analysis

GFP-marked glial cells were prepared from GFP-transgenic rat brain that ubiquitously expresses GFP from a chicken β-actin promoter (Hakamata et al., 2001). In this published study, GFP was detected in all cell types in the brain. The procedure for glial cell isolation was according to a published protocol (Pang et al., 2011). Briefly, postnatal day 1 pups were anesthetized on ice. Heads were removed with surgical scissors and transferred into a fresh 10 cm plate. Brain tissues were dissected out with a curved-tip forceps and collected in a 10 cm dish containing 10 ml cold HBSS. Cortices were isolated under a dissecting microscope and placed in a fresh 10 cm dish. Cortical tissues were cut into small pieces, re-suspended in 2 ml HBSS, and transferred to a 50 ml centrifuge tube. The dissection of cortical tissues was repeated twice. Small tissue pieces in 6 ml of HBSS were combined, to which 750 μl 10×Trypsin/EDTA and 750 μl of 10 mg/ml DNase I were added. The sample was vigorously agitated for 15 min in a 37° C. water bath to favor enzymatic digestion of the tissue. The tube was let stand for 5 min and 5 ml dissociated cells collected in a new 50-ml centrifuge tube containing the MEF media. The remaining undissociated tissue was trypsinized one more time with another 6 ml of HBSS containing 750 μl of 10×trypsin/EDTA and 750 μl of 10 mg/ml DNase I. Dissociated cells were filtered through a 100-μm nylon cell strainer and collected in a fresh 50-ml centrifuge tube. Dissociated glial cells were collected by centrifugation at 200 g. Supernatant was removed and the cells were re-suspended in culture media and seeded on a 10 cm tissue culture dish (at the density of cells from 2-3 cortices per 10 cm dish). The media were replaced daily until cells become confluent. Cells were split three times at 1:2 ratio with 0.25% Trypsin in order to remove any remaining neurons from the culture. Before co-culturing with MEF-derived neuronal-like cells, Tuj1 staining was performed to ensure no contaminating neurons.

Single cell patch clamp recordings were performed using an Axopatch 200B amplifier and pClamp 10.0 software (HEKA Elektronik, Lambrecht/Pfalz, Germany), as described (Ouyang et al., 2005). Under whole-cell voltage clamp conditions, membrane voltage was held at −70 mV with the pipette resistance of 4-6 MΩ. Test pulses in 80-ms duration were applied from −60 mV to +80 mV every 2 s. Action potentials were elicited by injecting 20-ms depolarizing currents with graded stimulus amplitudes under current clamp conditions. Standard external solution contains 150 mM NaCl, 5 mM KCl, 1 mM CaCl₂, 2 mM MgCl₂, 10 mM HEPES-pH 7.4 (pH adjusted with NaOH), and 10 mM glucose. Intracellular pipette solution contains 150 mM KCl, 5 mM NaCl, 1 mM MgCl₂, 2 mM EGTA, 1 mM MgATP, and 10 mM HEPES-pH 7.2 (pH adjusted with KOH). All experiments were performed at room temperature (20-22° C.).

RT-qPCR, Western Blotting, and Luciferase Assays

RNA was isolated with Trizol (Life Technology) following manufacturer's instructions, treated with DNase I (Promega), and reverse transcribed with Superscript III (Life Technology). Quantitative PCR (qPCR) was performed with Fast Start universal SYBR green master mix (Roche) along with gene specific primers on a real-time PCR machine (Applied Biosystems). PCR primers used in the present study are listed in Table 1. Statistical significance was determined by Students t-test based on triplicated experiments.

For analyses by Western blotting, total protein in 1×SDS loading buffer was first normalized based on quantification on NanoDrop (Thermo Scientific), and then resolved by 10% SDS-PAGE. Antibodies used in this study include Rabbit anti-PTBP1 (NT), Mouse anti-PTBP1 (monoclonal BB7) and Rabbit anti-PTBP2 (IS2), all of which are gifts of Douglass Black, Rabbit anti-PTBP2, a gift of Robert Darnell, Mouse anti-ACTB (Sigma), Rabbit anti-SCP1, a gift of Samuel L. Pfaff. Mouse anti-HDAC1 (Active Motif) and Mouse anti-EIF2C2 were purchased from Abnova.

Luciferase reporters were constructed by cloning the 3′UTR region of PTB regulated genes PCR-amplified from HEK293T genomic DNA into the Psicheck-2 vector between XhoI and Not I restriction sites. PCR primers used for constructing individual luciferase reporters are listed in Table 1. For transfection, cells were seeded in 24-well plates for 16 hrs and transfected using Lipofectamine 2000 (Life Technology) with a mix containing 20 ng reporter plasmid, 20 pmol miRNA mimics (Qiagen) or siRNAs (Dharmacon). Luciferase activity was measured 24 hrs post-transfection using the dual-luciferase reporter assay kit (Promega) on Veritas microplate luminometer (Promega).

RNA-Seq, CLIP-Seq, ChIP and Statistical Analysis of Data

RNA-seq was typically done after shRNA treatment for 72 hrs. Trizol-isolated RNA was enriched in two rounds for Poly(A⁺) RNA with paramagnetic oligo(dT), fragmented into ˜200 nt in length, converted to cDNA with Superscript III (Invitrogen), and subjected to deep sequencing. RNA-seq tags were mapped to the human genome (hg18) by using Tophat with parameters (-mate-inner-dist 150-solexa1.3-quals-max-multihits 10-microexon-search). The junction library was made from transcripts from UCSC RefGene and knownGene tables. RefGene transcripts were clustered by using NCBI Entrez GeneID, and treated as one gene to calculate gene expression. For each gene, only tags uniquely mapped and localized in exons or exon-exon junctions were counted.

Differential expressed genes were identified by using edgeR/DEGseq (Robinson et al., 2010; Wang et al., 2010) in combination with a fold-change cutoff as specified in the text. For example, at a threshold of FDR (Bonferroni corrected)<0.001 and >1.8-fold change, 538 down-regulated and 420 up-regulated genes were detected to be significantly differentially expressed upon PTB depletion in HeLa cells. Gene ontology category enrichment was assessed using GOrilla (http://cbl-gorilla.cs.technion.ac.il/) and DAVID online tools (http://david.abcc.ncifcr.gov/).

To determine PTB knockdown-induced switch of polyadenylation, we employed the MAPS technology as described (Fox-Walsh et al., 2011), which measures the tag count upstream of individual polyadenylation sites of expressed genes. For data analysis, we first removed sequences of adaptor and polyA tail from sequenced tags. To avoid false calls resulting from priming of internal A-rich regions, we scanned the genome for polyA-stretch defined as consecutive 8As, which were removed. We next adaptively clustered tags within a specific distance 30 nt along transcripts and sorted cluster intervals by the length and tag in a decreasing order. If a cluster contains a known 3′end within a 300 nt window, we used the end and then counted the number of reads in each cluster. Only cleavage sites that are supported by at least 10 reads were considered significant polyadenylation sites and used for subsequent analyses. A total of 6166 poly(A) sites was identified in Hela cells in the current analysis. To statistically detect transcripts that showed significant switch in polyadenylation in response to PTB knockdown, we defined the polyA switch ratio in order to measure the relative usage of competing sites within a transcript and then computer ratio changes in response to PTB knockdown. This analysis revealed 324 transcripts that show alternative polyadenylation sites. 14 of these transcripts showed PTB knockdown-induced shift from the distal to the proximal site. For global analysis, cumulative distribution was determined in both PTB expressing and PTB depleted cells. The plot was generated using R (http://www.r-project.org/) and Matlab (http://www.mathworks.com/). Two-sided Kolmogorov-Smirnov statistics (in the R package, http://cran.r-project.org/) was used to determine the significance of the shift in pair-wise comparison (Conover, 1971).

CLIP-seq was performed as previously described (Xue et al., 2009) with a mouse monoclonal anti-Ago2 antibody (also called EIF2C2). To eliminate redundancies from PCR amplification, all tags mapped to identical locations in the human genome were compressed to singles. Individual Ago2 tags after normalization according to total density between samples are plotted relative to the stop codon at the 3′ end of genes as described (Chi et al., 2009). To determine the distribution/distance of Ago2 tags relative to PTB peaks, we plotted the distribution of Ago2 tags in a 1 kb window around distinct genomic regions of PTB binding clusters. To compensate for differences in the number of reads in different samples, the number of tags at each position was divided by the total number of mapped tags in the two libraries constructed on cells before and after PTB knockdown. Tag density heat maps were created by first using custom Python scripts to generate tag densities matrix by dividing each region into 5 nt bins for each PTB cluster in genic 3′UTR region and then visualized using Java TreeView (http://jtreeview.sourceforge.net), as described (Saldanha, 2004). The observed density in the heap map was ranked by tag counts in 1 kb windows around peak center (from bottom to top). The sum of Ago2 reads at each position was calculated and displayed as fraction value (dark line).

To determine the functional correlation between PTB/microRNA interplay and gene expression, we focused on PTB and Ago2 binding sites at the 3′UTR. Cumulative distribution was individually determined in each of 5 categories as described in the text. Plots were generated using R (http://www.r-project.org/) and Matlab (http://www.mathworks.com/). Two-sided Kolmogorov-Smirnov statistics (in the R package, http://cran.r-project.org/) was used to determine the significance of the shift in each pair-wise comparison (Conover, 1971).

ChIP was performed with a rabbit anti-REST antibody purchased from Milipore (07-579). Briefly, MEF cells were crosslinked with 1% formaldehyde for 10 min at room temperature, which was quenched with 100 mM Tris-Cl (pH 9.4), 10 mM DTT for 10 min on ice. Cell pellets were lysed with cell lysis buffer (1% SDS, 10 mM EDTA, 50 mM Tris-Cl, pH8.1, 1×Protease Inhibitor cocktail) for 10 min on ice. The lysate was sonicated five times for 10 second each at the maximum setting. The sonicated chromatin was checked on 1% agarose gel to make sure sheared chromatin in a range of 200-300 bp. The sonicated lysate was centrifuged at 14000 rpm for 10 min at 4° C. Soluble chromatin was then 1:10 diluted in dilution buffer (1% Triton X-100, 2 mM EDTA, 150 mM NaCl, 20 mM Tris-Cl, pH8.1, 1×Protease Inhibitor cocktail). Equal volumes of diluted chromatin were taken to two Eppendorf tubes to which 5 μg of rabbit normal IgG or rabbit anti-REST were added. The reaction was incubated with periodic shake overnight at 4° C. 35 μl protein G magnetic beads were then added to each tube and the reaction continued with periodic shake for another 4 hours at 4° C. At the end of the reaction, the beads were washed twice with TSE 1 (0.1% SDS, 1% Triton X-100, 2 mM EDTA, 150 mM NaCl, 20 mM Tris-Cl, pH8.1), with TSE II (0.1% SDS, 1% Triton X-100, 2 mM EDTA, 500 mM NaCl, 20 mM Tris-Cl, pH8.1), and finally three times with TE buffer. Lastly, beads were eluted twice with TE buffer plus 1% SDS at 65° C. for 10 min. The eluents and input samples were reverse crosslinked overnight at 65° C. DNA fragments were purified with QIAquick spin gel extraction kit and qPCR was performed with gene specific primer pairs.

Analysis of RNA Secondary Structure by RNase Protection and in-Line Probing

For RNA foot-printing assay, GNPDA1 RNA was generated by T7 in vitro transcription. RNA was 5′-labeled using T4 PNK with γ-³²P ATP. The RNA was purified by cutting specific labeled band from 7M urea-8% polyacrylamide gel and eluted 3 hrs with G-50 buffer (300 mM NaoAC, 1 mM EDTA, 0.05% SDS). The RNA structure was probed with RNase T1 and RNase V1 following the manual of Ambion/Life Technology. Briefly, 20,000 cpm of end-labeled RNA and 3 μg yeast tRNA were incubated with 0.1 U RNase T1 or 0.01 U RNase V1 in 1×RNA structure probing buffer for 15 min at room temperature. After the addition of 20 μl of Inactivation/Precipitation buffer to the tube and incubation at −20° C. for 15 min, samples were centrifuged at 13,200 rpm for 15 min, supernatant aspirated, and pellet washed with 70% ethanol. The pellet was dissolved in 7 μl of acrylamide gel loading buffer, denatured at 95° C. for 5 min, and 3 μl was fractionated on 8% acrylamide/7M urea gel. For RNA sequencing reaction, the same amount of end-labeled RNA and tRNA were incubated with 0.1 U RNase T1 or 0.01 U RNase V1 in 1× sequencing buffer at 50° C. for 5 min. Single nucleotide RNA ladders were generated by incubating similar amounts of 5′-end labeled RNA and tRNA with RNA hydrolysis buffer (50 mM sodium carbonate pH-9.2, 1 mM EDTA) at 95° C. for 12 min. To probe PTB-RNA interactions, His-tagged PTB4 protein was added to the RNA structure buffer to a final concentration of 2 μM and the reaction was incubated at 30° C. for 10 min after which the same amounts of RNase T1 or Rnase V1 were added to probe structural changes.

For in-line probing, 30,000 cpm of 5′-labeled RNA and 1 μg yeast tRNA were first incubated with varying amounts of His-tagged PTB4 protein in 1× In-line reaction buffer (50 mM Tris-HCl, pH-8.3, 20 mM MgCl₂, 100 mM KCl) at 30° C. for 10 min. The reaction was further incubated at 23° C. for 40 h. The reaction was quenched by adding 2× colorless gel-loading solution and 5 μl was fractionated on 8% acrylamide/7M urea gel.

### REFERENCES

Chi, S. W., Zang, J. B., Mele, A., and Darnell, R. B. (2009). Argonaute HITS-CLIP decodes microRNA-mRNA interaction maps. Nature 460, 479-486.
Conover, W. (1971). Practical nonparametric statistics. New York, John Wiley & Son, 309-314.
Fox-Walsh, K., Davis-Turak, J., Zhou, Y., Li, H., and Fu, X. D. (2011). A multiplex RNA-seq strategy to profile poly(A+) RNA: application to analysis of transcription response and 3′ end formation. Genomics 98, 266-271.
Hakamata, Y., Tahara, K., Uchida, H., Sakuma, Y., Nakamura, M., Kume, A., Murakami, T., Takahashi, M., Takahashi, R., Hirabayashi, M., et al. (2001). Green fluorescent protein-transgenic rat: a tool for organ transplantation research. Biochem Biophys Res Commun 286, 779-785.
Huang, C., Xie, M. H., Liu, W., Yang, B., Yang, F., Huang, J., Huang, J., Wu, Q., Fu, X. D., and Zhang, Y. (2011). A structured RNA in hepatitis B virus post-transcriptional regulatory element represses alternative splicing in a sequence-independent and position-dependent manner. Febs J.
Makeyev, E. V., Zhang, J., Carrasco, M. A., and Maniatis, T. (2007). The MicroRNA miR-124 promotes neuronal differentiation by triggering brain-specific alternative pre-mRNA splicing. Mol Cell 27, 435-448.
Ouyang, K., Wu, C., and Cheng, H. (2005). Ca(2+)-induced Ca(2+) release in sensory neurons: low gain amplification confers intrinsic stability. J Biol Chem 280, 15898-15902.
Pang, Z. P., Yang, N., Vierbuchen, T., Ostermeier, A., Fuentes, D. R., Yang, T. Q., Citri, A., Sebastiano, V., Marro, S., Sudhof, T. C., et al. (2011). Induction of human neuronal cells by defined transcription factors. Nature 467, 220-223.
Robinson, M. D., McCarthy, D. J., and Smyth, G. K. (2010). edgeR: a Bioconductor package for differential expression analysis of digital gene expression data. Bioinformatics 26, 139-140.
Saldanha, A. J. (2004). Java Treeview-extensible visualization of microarray data. Bioinformatics 20, 3246-3248.
Wang, L., Feng, Z., Wang, X., Wang, X., and Zhang, X. (2010). DEGseq: an R package for identifying differentially expressed genes from RNA-seq data. Bioinformatics 26, 136-138.
Xue, Y., Zhou, Y., Wu, T., Zhu, T., Ji, X., Kwon, Y. S., Zhang, C., Yeo, G., Black, D. L., Sun, H., et al. (2009). Genome-wide analysis of PTB-RNA interactions reveals a strategy used by the general splicing repressor to modulate exon inclusion or skipping. Mol Cell 36, 996-1006.

Sequentially Knocking Down PTB and nPTB to Generate Functional Human Neuronal Cells

In alternative embodiments, the invention provides methods for generating a fully functional human mature neuron from non-neuronal cells, e.g., fibroblasts, or neuronal precursors, such as ectodermal or neuronal stem cells or undifferentiated cells, comprising the sequential knocking down of first Polypyrimidine Tract Binding protein (PTB) and then nPTB (the “neuronal PTB” homolog, or nPTB).

In mouse cells, it appears that PTB knockdown is sufficient to drive cells to fully functional mature neurons. However, this does not seem to be the case on human fibroblasts, especially those aged individuals. PTB knockdown can potentially induce the neuronal morphology and early neuronal marks, such Tuj1, but those human cell-derived neurons lack mature neuron marks. This may explain why human cells are much harder to reprogram into functional neurons.

PTB has a homolog known as nPTB in mammalian genomes (the “neuronal PTB” homolog, or nPTB). Published studies reveal a sequential switch in PTB and nPTB expression during neuronal induction and maturation: In neuroblasts, PTB but not nPTB is expressed; during early neuronal induction, PTB expression is diminished and nPTB is induced; in mature neurons, the expression of both PTB and nPTB is diminished. Based on this temporal pattern of PTB and nPTB expression, we hypothesized that PTB may function as a key barrier for initial neuronal induction, while nPTB may act as another key barrier for neuronal maturation.

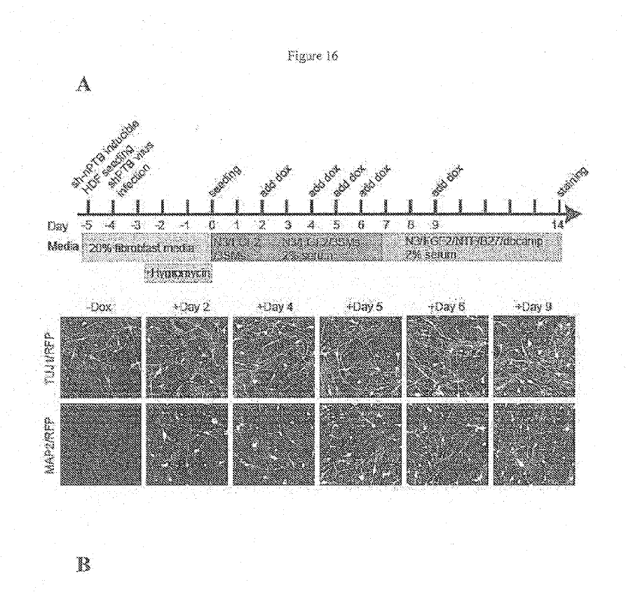

We performed the experiment to test this hypothesis by sequentially knocking down PTB and nPTB. This is critical because simultaneous knockdown of PTB and nPTB will cause a cell lethal phenotype. As shown in the FIG. 16, PTB knockdown followed by nPTB knockout efficiently converted human fibroblasts to neurons with mature neuronal marks, such as MAP2. It was demonstrated that nPTB has to be knocked down 4 days or later to achieve this phenotype. Accordingly, this exemplary embodiment provides methods for converting non-neuronal human cells to functional neurons for regenerative medicine.

A number of embodiments of the invention have been described. Nevertheless, it will be understood that various modifications may be made without departing from the spirit and scope of the invention. Accordingly, other embodiments are within the scope of the following claims.

1. An in vitro, ex vivo or in vivo method for trans-differentiating, re-differentiating or re-programming a mammalian cell to a neuronal cell, comprising:
(a) (i) providing a composition or compound for:
reducing or lowering the level of expression of or activity of or inactivating a Polypyrimidine Tract Binding protein (PTB) gene, message or protein;
reducing or lowering the level of expression of or activity of or inactivating a “neuronal PTB homologue”, or nPTB, gene, message or protein; or
reducing or lowering the level of expression of or activity of or inactivating an RE1-Silencing Transcription factor (REST; also known as Neuron-Restrictive Silencer Factor, or NRSF) complex;
(ii) providing a non-neuronal mammalian cell; and
(iii) contacting in vitro, ex vivo or in vivo the composition or compound with the non-neuronal mammalian cell in an amount effective to cause the trans-differentiating, re-differentiating or re-programming of the mammalian cell to a neuronal cell;
(b) identifying and/or isolating the trans-differentiated, re-differentiated or re-programmed mammalian cell.

2. The method of claim 1, wherein the mammalian cell is selected from the group consisting of a human cell, a non-human primate cell, a monkey cell, a mouse cell, a rat cell, a guinea pig cell, a rabbit cell, a hamster cell, a goat cell, a bovine cell, an equine cell, an ovine cell, a canine cell and a feline cell.

3. The method of claim 1, wherein the composition or compound comprises a liquid or aqueous formulation, a vesicle, liposome, nanoparticle or nanolipid particle.

4. The method of claim 3, wherein the mammalian cell is contacted with the composition or compound in an amount effective to cause the trans-differentiation or re-programming of the mammalian cell to a neuronal cell.

5. The method of claim 1, wherein the mammalian cell before trans-differentiation or re-programming is an adult stem cell, an embryonic stem cell, a somatic stem cell, an adipose-derived stem cell (ASC), a stem cell derived from an epithelial cell or tissue, a hematopoietic stem cell, a mammary stem cell, a mesenchymal stem cell, a neural stem cell, an olfactory adult stem cell, a spermatogonial progenitor cell, a dental pulp-derived stem cell, a cancer stem cell, an adult somatic cell, an adult germ cell, a hematopoietic cell, a lymphocyte, a macrophage, a T cell, a B cell, a nerve cell, a neural cell, a glial cell, an astrocyte, a muscle cell, a cardiac cell, a liver cell, a hepatocyte, a pancreatic cell, a fibroblast cell, a connective tissue cell, a skin cell, a melanocyte, an adipose cell, an exocrine cell, a dermal cell, a keratinocyte, a retinal cell, a Muller cell, a mucosal cell, an esophageal cell, an epidermal cell, a bone cell, a chondrocyte, an osteoblast, an osteocyte, a prostate cell, an embryoid body cell, an ovary cell, a testis cell, an adipose tissue (fat) cell, or a cancer cell.

6. The method of claim 1, wherein the mammalian cell is cultured for between about one hour to two days.

7. (canceled)

8. The method of claim 1, further comprising contacting the mammalian cells with a cytokine that has a trans-differentiation or re-programming effect on the mammalian cell.

9. (canceled)

10. The method of claim 1, wherein identifying and/or isolating the trans-differentiated or re-programmed cell is by
(a) a negative selection of cells still expressing a non-neuronal cell marker,
(b) by fluorescent activated cell sorting (FACS),
(c) by affinity column chromatography,
(d) by identification and/or isolation of plasma membrane proteins by mass spectography or chromatography, or
(e) by determining the presence or absence of a message (mRNA, transcript) determinative of an undifferentiated or neuronal cell phenotype.

11. The method of claim 1, further comprising implanting the trans-differentiated or re-programmed mammalian cell in or into a vessel, tissue or organ.

12. The method of claim 1, further comprising implanting the trans-differentiated or re-programmed mammalian cell in or into an individual in need thereof.

13. A trans-differentiated or re-programmed cell made by the method of claim 1.

14. The trans-differentiated or re-programmed cell of claim 13, wherein the mammalian cell is selected from the group consisting of a human cell, a non-human primate cell, a monkey cell, a mouse cell, a rat cell, a guinea pig cell, a rabbit cell, a hamster cell, a goat cell, a bovine cell, an equine cell, an ovine cell, a canine cell and a feline cell.

15. (canceled)

16. (canceled)

17. The method of claim 12, wherein the individual suffers from a neurodegenerative disease or injury, or neurodegenerative condition selected from the group consisting of Alzheimer's disease (AD), Parkinson's disease (PD), Huntington's disease (HD), a Polyglutamine (PolyQ) Disease, Amyotrophic lateral sclerosis (ALS), traumatic brain injury (TBI), Chronic traumatic encephalopathy (CTE), a paralysis, a stroke and an ischemic injury.

18. A formulation, a product of manufacture, an implant, an artificial organ or a tissue, or a kit, comprising a trans-differentiated or re-programmed cell of claim 13.

19. The method of claim 1, wherein the composition or compound is selected from the group consisting of a protein, a peptide, an antibody, a nucleic acid, an antisense or miRNA nucleic acid, or a small molecule.

20. The method of claim 19, wherein the antisense or miRNA nucleic acid comprises a neuronal-specific miR-124.

21. The method of claim 1, wherein the method comprises the sequential reducing or lowering of expression or activity of first PTB, and then nPTB, in the mammalian cell.

22. The method of claim 1, wherein the mammalian cell to be trans-differentiated, re-differentiated or re-programmed is a fibroblast or glial cell.

23. The method of claim 8, wherein the cytokine is selected from the group consisting of a transforming growth factor-beta (TGF-beta), interleukin-18, adipose complement-related protein, interferon-γ and any composition thereof.
